(19) United States
(12) Patent Application Publication
(10) Pub. No.: US 2004/0209937 A1
(43) Pub. Date: Oct. 21, 2004

(54) Treatment of excessive osteolysis with indolinone compounds

(75) Inventors: Lesley Murray, San Jose, CA (US); Anne-Marie O'Farrell, Menlo Park, CA (US); Tinya Abrams, Richmond, CA (US)

Correspondence Address: FOLEY AND LARDNER, SUITE 500, 3000 K STREET NW, WASHINGTON, DC, 20007, US

(73) Assignee: SUGEN, INC.

(21) Appl. No.: 10/780,917

(22) Filed: Feb. 19, 2004

Related U.S. Application Data
(60) Provisional application No. 60/448,861, filed on Feb. 24, 2003.

(51) Int. Cl. A61K 31/403

(52) U.S. Cl. 514/414

(57) ABSTRACT

Compounds of Formula I and Formula II, as described herein, are useful for treating excessive osteolysis, by inhibiting M-CSF mediated osteoclast development. The compounds also are useful for inhibiting phosphorylation of CSF1R, and for treating cancers that express CSF1R.

### BACKGROUND OF THE INVENTION

Bone is a dynamic tissue, subject to a constant remodeling process that operates to maintain skeletal strength and health. This remodeling process entails two phases: an osteolysis phase and an osteogenesis phase. In osteolysis, osteoclast cells invade bone and erode it by releasing a cocktail of acids and enzymes that dissolve collagen and minerals. This creates a small cavity in the bone. In osteogenesis, osteoblast cells deposit new collagen and minerals into the cavity. When osteolysis and osteogenesis are in balance, no net change in bone mass results. Each year, the bone remodeling process replaces approximately 20% of a healthy individual's bone throughout the skeleton.

In certain disease states, osteolysis is more active than osteogenesis, resulting in a net loss of bone. Such excessive osteolysis may occur in localized areas of the skeleton or more broadly throughout the skeleton. Regardless, bone loss has serious health consequences, including fractures, hypercalcemia, nerve compression syndromes, deformity and pain.

One particularly serious cause of localized excessive osteolysis is cancer metastasis to bone. Cancer cells often secrete factors, such as macrophage colony stimulating factor (M-CSF), that promote osteoclast development and activity. When such cancers establish themselves in bone, they promote extensive osteolytic damage. Such tumor-associated osteolysis coincides with many types of malignancies, including hematological malignancies (e.g., myeloma and lymphoma) and solid tumors (e.g., breast, prostate, lung, renal and thyroid). In fact, 70% of patients dying of breast cancer have bone metastases, and bone is the most common site of first distant recurrence for breast cancer. Cancer patients with bone metastases may survive for several years, which highlights the need for therapies that reduce the effects of bone metastases.

When excessive osteolysis occurs throughout broad areas of the skeleton, it falls under the generic description osteoporosis. Common types of osteoporosis include age-related, post-menopausal, glucocorticoid-induced, diabetes-associated and disuse osteoporosis. Worldwide, osteoporosis presents a staggering problem. In the United States alone, millions of individuals suffer from the disease and its attendant pain, deformities and debilitating fractures.

Osteoclasts, the cells that mediate excessive osteolysis, operate under the control of numerous cytokines and growth factors (1, 2). They are multinucleated cells that derive from monocytic precursors (3,4). Differentiation of the monocytic precursors into osteoclasts is a complex process that requires both M-CSF and RANKL (receptor activator of the NF-κB ligand) (3,4). These factors, and all other cellular and humoral requirements for osteoclast differentiation exist in the micro-environment of a skeletal metastasis (17).

Macrophages, which are related to osteoclasts, are a major component of the host cellular response to cancers, and can contribute to tumor growth. In particular, macrophages, as well as tumor cells, secrete M-CSF, a key cytokine for development of osteoclasts from monocyte precursors (18-22). Macrophages, as well as monocytes and some tumor cells, also express M-CSF receptors (23-28).

Inhibiting osteoclast development and function is a desirable approach to treating excessive osteolysis. However, the currently available substances that do so have limited utility, and often cause significant side effects.

Calcitonin, a peptide hormone secreted by the thyroid in response to elevated serum calcium, is a well-characterized inhibitor of osteoclast formation and function (5). However, chronic exposure to calcitonin leads to loss of its inhibitory effects on osteoclasts, through down regulation of the calcitonin receptor mRNA and calcitonin receptors on the surface of osteoclasts (6). Additionally, because calcitonin is a protein, it cannot be taken orally, as it would be digested before it could work. While it does not affect other organs or systems in the body, injectable calcitonin may cause an allergic reaction and unpleasant side effects including flushing of the face and hands, urinary frequency, nausea and a skin rash.

Like calcitonin, TGF-β and echistatin, a snake venom, block osteolysis in vitro (7). However, TGF-β and echistatin lack specificity, and echistatin blocks platelet adhesion, thereby potentially causing life-threatening bleeding.

Monoclonal antibodies that bind antigens expressed on osteoclast cells also can block osteolysis (8-10). However, such antibodies can induce an immune response in patients.

Bisphosphonates also inhibit osteoclast activity (11), and extensive data exists regarding their use. Despite their benefits, bisphosphonates are poorly absorbed from the gastrointestinal tract and often induce gastrointestinal discomfort. Moreover, bisphosphonates remain in the bone for years, creating a potential of blocking normal bone repair mechanisms if too much is taken for too long.

Immune cell products such as interferon-gamma (IFN-γ), interferon-alpha (IFN-α), oncostatin M, and taxol, suramine and nitric oxide inhibit osteoclast activity as well (12-14). However, all of these agents have significant side effects that limit their utility. Interferons can induce flu-like illness, taxol and suramine frequently have severe toxicities associated with gastrointestinal and/or hematopoietic side effects, and nitric oxide can induce vasodilation and low blood pressure.

Estradiol is yet another well-known inhibitor of osteolysis (15) that induces apoptosis, or programmed cell death, in osteoclasts. Similarly, retinoic acid inhibits osteolysis (16). Unfortunately, these compounds also cause systemic effects that make them less acceptable. For example, long-term estrogen therapy poses an increased risk of breast, uterine, and ovarian cancer. Additionally, estradiol therapy can cause vaginal bleeding, breast tenderness, mood disturbances and gallbladder disease. Selective estrogen receptor modulators (SERMS) mimic estrogens in some tissues and anti-estrogens in others. They have the advantage of causing fewer unwanted side effects, but still provoke hot flashes and deep vein thrombosis in some patients.

Thus, a continuing need exists for effective and practical treatments for excessive osteolytic conditions. Based on this continuing need, the present inventors developed a method of treating excessive osteolytic conditions by administering an effective amount of a compound of formula I or II, which are described below.

### SUMMARY OF THE INVENTION

One embodiment of the invention relates to a method of treating excessive osteolysis by administering an effective amount of a compound of Formula I:

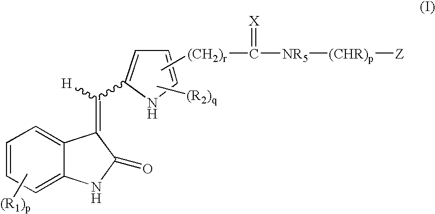

wherein

R is independently H, OH, alkyl, aryl, cycloalkyl, heteroaryl, alkoxy, heterocyclic and amino; each R₁ is independently selected from the group consisting of alkyl, halo, aryl, alkoxy, haloalkyl, haloalkoxy, cycloalkyl, heteroaryl, heterocyclic, hydroxy,

—C(O)—R₈, —NR₉R₁₀, —NR₉C(O)—R₁₂ and —C(O)NR₉R₁₀;

each R₂ is independently selected from the group consisting of alkyl, aryl, heteroaryl, —C(O)—R₈, and SO₂R″, where R″ is alkyl, aryl, heteroaryl, NR₉N₁₀ or alkoxy;

each R₅ is independently selected from the group consisting of hydrogen, alkyl, aryl, haloalkyl, cycloalkyl, heteroaryl, heterocyclic, hydroxy, —C(O)—R₈ and (CHR)ᵣR₁₁;

X is O or S;

p is 0-3;

q is 0-2;

r is 0-3;

R₈ is selected from the group consisting of —OH, alkyl, aryl, heteroaryl, alkoxy, cycloalkyl and heterocyclic;

R₉ and R₁₀ are independently selected from the group consisting of H, alkyl, aryl, aminoalkyl, heteroaryl, cycloalkyl and heterocyclic, or R₉ and R₁₀ together with N may form a ring, where the ring atoms are selected from the group consisting of C, N, O and S;

R₁₁ is selected from the group consisting of —OH, amino, monosubstituted amino, disubstituted amino, alkyl, aryl, heteroaryl, alkoxy, cycloalkyl and heterocyclic;

R₁₂ is selected from the group consisting of alkyl, aryl, heteroaryl, alkoxy, cycloalkyl and heterocyclic;

Z is OH, O-alkyl, or —NR₃R₄, where R₃ and R₄ are independently selected from the group consisting of hydrogen, alkyl, aryl, heteroaryl, cycloalkyl, and heterocyclic, or R₃ and R₄ may combine with N to form a ring where the ring atoms are selected from the group consisting of CH₂, N, O and S or

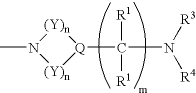

wherein Y is independently CH₂, O, N or S,

Q is C or N;

n is independently 0-4; and

m is 0-3;

or a salt thereof, to a patient in need of such treatment.

In a preferred embodiment, the compound administered to the patient is a compound of Formula II:

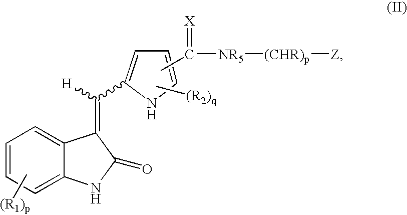

where the variables are as previously defined.

In another embodiment of the invention, R₁ is halo (e.g., F and Cl) and Z is —NR₃R₄ wherein R₃ and R₄ are independently selected from alkyl and hydrogen in Formula I or II as administered to a patient in need thereof.

In another embodiment, Z of Formula I or II is —NR₃R₄, wherein R₃ and R₄ form a morpholine ring.

In another embodiment, Z of Formula I or II is:

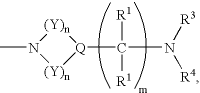

wherein each Y is CH₂, each n is 2, m is 0 and R₃ and R₄ form a morpholine ring.

In any of the previously recited embodiments, preferably R₂ is methyl and q is 2, wherein the methyls are bonded at the 3 and 5 positions of Formula I or II.

In a particular embodiment of the invention, the compound administered is selected from the group consisting of

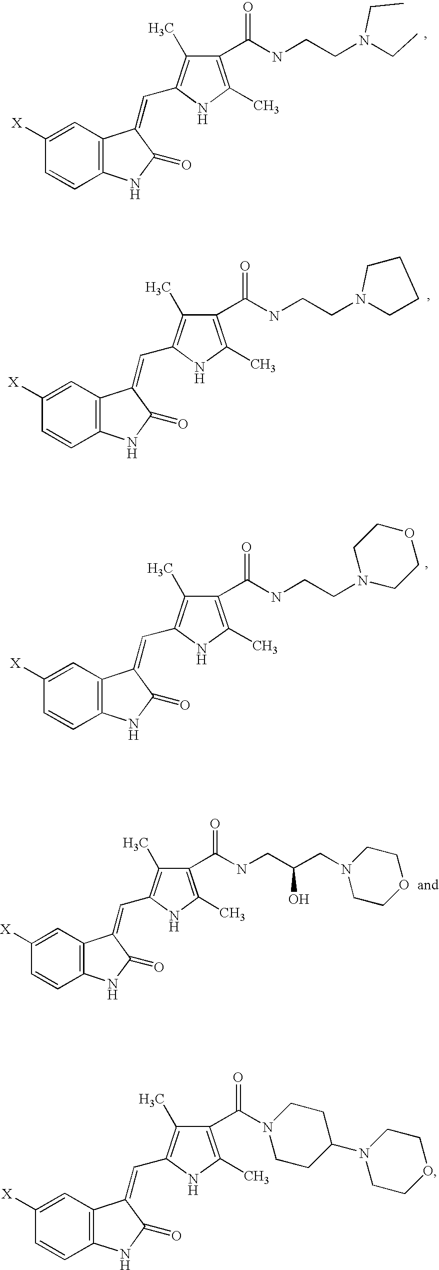

wherein X is F, Cl, I or Br. In a preferred embodiment, X is F.

In another particular embodiment of the invention, the compound of formula I is selected from the group consisting of:

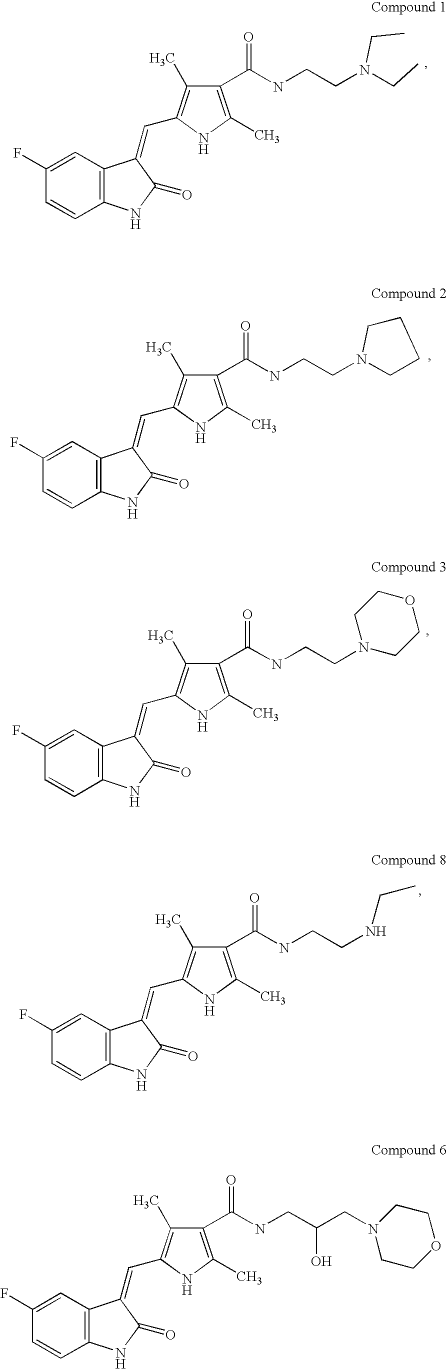

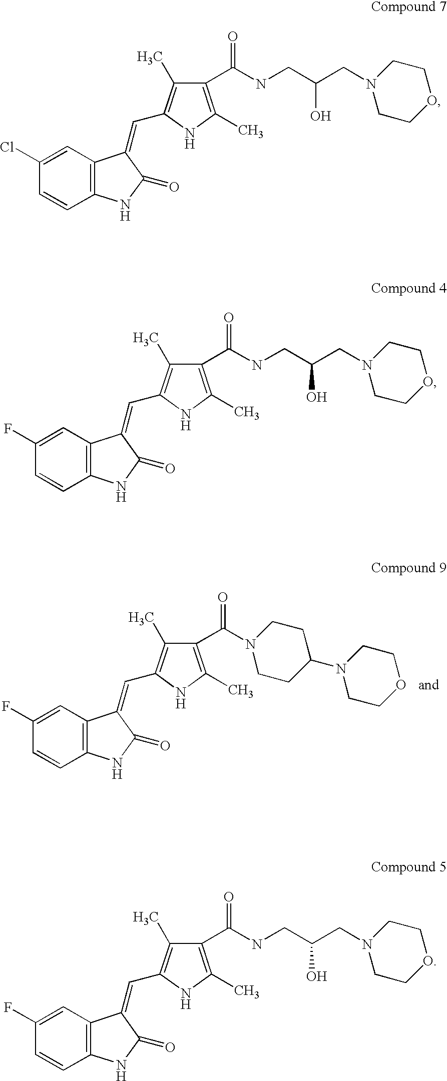

In certain embodiments of the invention, the patient suffering from excessive osteolysis has osteoporosis, cancer that has metastasized to bone, cancer that secretes M-CSF, and/or is post-menopausal.

Yet another embodiment of the invention relates to a method of treating a cancer that expresses CSF1R, the M-CSF receptor, by administering an inhibitory amount of a compound of Formula I or Formula II, as described above, to a patient. In one embodiment, the cancer is determined to express CSF1R prior to administration of a compound of Formula I or Formula II.

Another embodiment of the invention pertains to a method of inhibiting phosphorylation of CSF1R by administering an inhibitory amount of a compound of Formula I or Formula II, as described above, to a patient.

### BRIEF DESCRIPTION OF THE DRAWINGS

FIG. 1 shows a Western Blot demonstrating that compound 1 of the invention inhibits phosphorylation of M-CSF receptors.

FIG. 2a shows a graph demonstrating that compound 1 of the invention inhibits osteoclast development in a dose-dependent manner in vitro.

FIG. 2b shows a graph demonstrating that compound 1 of the invention inhibits an early stage of osteoclast development, but not later stages.

FIG. 3 depicts, via bioluminescence, that compound 1 of the invention inhibits the growth of breast cancer metastases in vivo.

FIG. 4 is a graph demonstrating that mice with breast cancer metastases to bone exhibit less osteolysis, as measured by pyridinoline levels, when treated with compound 1.

### DETAILED DESCRIPTION OF THE INVENTION

The present inventors have made the surprising discovery that chemical compounds having the structure of Formula I and Formula II, as set forth herein, inhibit phosphorylation of the M-CSF receptor, CSF1R. Moreover, the inventors have discovered that the compounds inhibit osteoclast development in vivo and significantly decrease osteolysis associated with tumor metastases to bone.

Accordingly, the compounds of Formula I and Formula II are useful in the treatment of patients with excessive osteolysis. In this context, “osteolysis” refers to a breakdown of bone by cells that secrete acids and/or enzymes. Osteoclasts are a primary example of such cells, but the invention embraces inhibition of osteolysis mediated by other cell types as well, including tumor cells and osteoclast precursors. “Excessive osteolysis” refers to an imbalance between osteolytic and osteogenetic activities that results in a net loss of bone, either locally or systemically.

One embodiment of the invention relates to a method of treating excessive osteolysis that comprises administering an effective amount of a compound of Formula I:

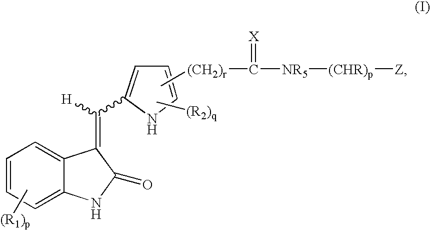

wherein

R is independently H, OH, alkyl, aryl, cycloalkyl, heteroaryl, alkoxy, heterocyclic and amino;

each R₁ is independently selected from the group consisting of alkyl, halo, aryl, alkoxy, haloalkyl, haloalkoxy, cycloalkyl, heteroaryl, heterocyclic, hydroxy, —C(O)—R₈, —NR₉R₁₀, —NR₉C(O)—R₁₂ and —C(O)NR₉R₁₀;

each R₂ is independently selected from the group consisting of alkyl, aryl, heteroaryl, —C(O)—R₈ and SO₂R″, where R″ is alkyl, aryl, heteroaryl, NR₉N₁₀ or alkoxy;

each R₅ is independently selected from the group consisting of hydrogen, alkyl, aryl, haloalkyl, cycloalkyl, heteroaryl, heterocyclic, hydroxy, —C(O)—R₈ and (CHR)ᵣR₁₁;

X is O or S;

p is 0-3;

q is 0-2;

r is 0-3;

R₈ is selected from the group consisting of —OH, alkyl, aryl, heteroaryl, alkoxy, cycloalkyl and heterocyclic;

R₉ and R₁₀ are independently selected from the group consisting of H, alkyl, aryl, aminoalkyl, heteroaryl, cycloalkyl and heterocyclic, or R₉ and R₁₀ together with N may form a ring, where the ring atoms are selected from the group consisting of C, N, O and S;

R₁₁ is selected from the group consisting of —OH, amino, monosubstituted amino, disubstituted amino, alkyl, aryl, heteroaryl, alkoxy, cycloalkyl and heterocyclic

R₁₂ is selected from the group consisting of alkyl, aryl, heteroaryl, alkoxy, cycloalkyl and heterocyclic;

Z is OH, O-alkyl, or —NR₃R₄, where R₃ and R₄ are independently selected from the group consisting of hydrogen, alkyl, aryl, heteroaryl, cycloalkyl, and heterocyclic, or R₃ and R₄ may combine with N to form a ring where the ring atoms are selected from the group consisting of CH₂, N, O and S or

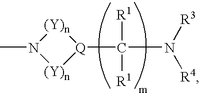

wherein Y is independently CH₂, O, N or S,

Q is C or N;

n is independently 0-4; and

m is 0-3;

or a salt thereof, to a patient in need of such treatment.

In a preferred embodiment, the compound administered to the patient is a compound of Formula II:

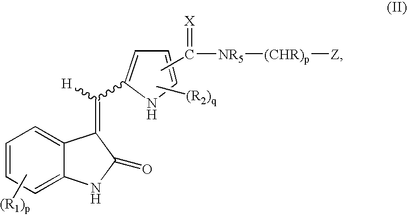

where the variables are as previously defined.

In another embodiment of the invention, R₁ is halo (e.g., F and Cl) and Z is —NR₃R₄ wherein R₃ and R₄ are independently H or alkyl in Formula I or II as administered to a patient in need thereof.

In another embodiment, Z of Formula I or II is —NR₃R₄ wherein R₃ and R₄ form a morpholine ring.

In another embodiment, Z of Formula I or II is:

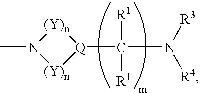

wherein each Y is CH₂, each n is 2, m is 0 and R₃ and R₄ form a morpholine ring.

In a particular embodiment of the invention, the compound administered is selected from the group consisting of

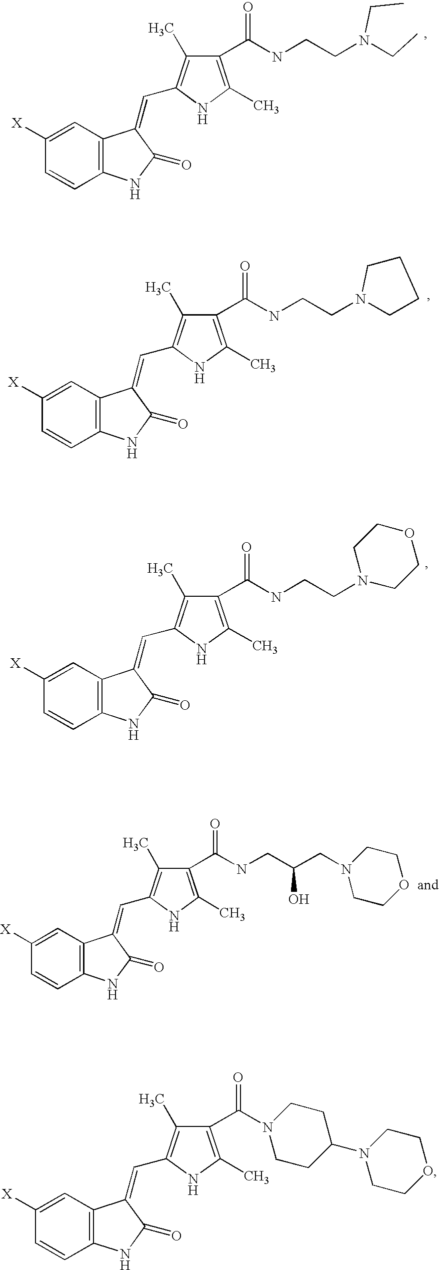

wherein X is F, Cl, I or Br. In a preferred embodiment, X is F.

In another embodiment of the invention, the therapeutic method involves administering to a patient having excessive osteolysis an effective amount of a compound selected from the group consisting of:

5-(5-Fluoro-2-oxo-1,2-dihydro-indol-3-ylidenemethyl)-2,4-dimethyl-1H-pyrrole-3-carboxylic acid (2-diethylamino-ethyl)-amide (compound 1);

5-(5-Fluoro-2-oxo-1,2-dihydro-indol-3-ylidenemethyl)-2,4-dimethyl-1H-pyrrole-3-carboxylic acid (2-pyrrolidin-1-yl-ethyl)-amide (compound 2);

5-(5-Fluoro-2-oxo-1,2-dihydro-indol-3-ylidenemethyl)-2,4-dimethyl-1H-pyrrole-3-carboxylic acid (2-morpholin-4-yl-ethyl)-amide (compound 3);

(S)-5-(5-Fluoro-2-oxo-1,2-dihydro-indol-3-ylidenemethyl)-2,4-dimethyl-1H-pyrrole-3-carboxylic acid (2-hydroxy-3-morpholin-4-yl-propyl)-amide (compound 4);

(R)-5-(5-Fluoro-2-oxo-1,2-dihydro-indol-3-ylidenemethyl)-2,4-dimethyl-1H-pyrrole-3-carboxylic acid (2-hydroxy-3-morpholin-4-yl-propyl)-amide (compound 5);

5-(5-Fluoro-2-oxo-1,2-dihydro-indol-3-ylidenemethyl)-2,4-dimethyl-1H-pyrrole-3-carboxylic acid (2-hydroxy-3-morpholin-4-yl-propyl)-amide (compound 6);

5-(5-Chloro-2-oxo-1,2-dihydro-indol-3-ylidenemethyl)-2,4-dimethyl-1H-pyrrole-3-carboxylic acid (2-hydroxy-3-morpholin-4-yl-propyl)-amide (compound 7);

5-(5-Fluoro-2-oxo-1,2-dihydro-indol-3-ylidenemethyl)-2,4-dimethyl-1H-pyrrole-3-carboxylic acid (2-ethylamino-ethyl)-amide (compound 8); and

3-[3,5-dimethyl-4-(4-morpholin-4-yl-piperidine-1-carbonyl)-1H-pyrrol-2-methylene]-5-fluoro-1,3-dihydro-indol-2-one (compound 9).

To clearly set forth the compounds of Formula I and II, useful in the inventive method, the following definitions are provided.

“Alkyl” refers to a saturated aliphatic hydrocarbon radical including straight chain and branched chain groups of 1 to 20 carbon atoms (whenever a numerical range; e.g. “1-20”, is stated herein, it means that the group, in this case the alkyl group, may contain 1 carbon atom, 2 carbon atoms, 3 carbon atoms, etc. up to and including 20 carbon atoms). Alkyl groups containing from 1 to 4 carbon atoms are referred to as lower alkyl groups. When said lower alkyl groups lack substituents, they are referred to as unsubstituted lower alkyl groups. More preferably, an alkyl group is a medium size alkyl having 1 to 10 carbon atoms e.g., methyl, ethyl, propyl, 2-propyl, n-butyl, iso-butyl, tert-butyl, pentyl, and the like. Most preferably, it is a lower alkyl having 1 to 4 carbon atoms e.g., methyl, ethyl, propyl, 2-propyl, n-butyl, iso-butyl, or tert-butyl, and the like. The alkyl group may be substituted or unsubstituted. When substituted, the substituent group(s) is preferably one or more, more preferably one to three, even more preferably one or two substituent(s) independently selected from the group consisting of halo, hydroxy, unsubstituted lower alkoxy, aryl optionally substituted with one or more groups, preferably one, two or three groups which are independently of each other halo, hydroxy, unsubstituted lower alkyl or unsubstituted lower alkoxy groups, aryloxy optionally substituted with one or more groups, preferably one, two or three groups which are independently of each other halo, hydroxy, unsubstituted lower alkyl or unsubstituted lower alkoxy groups, 6-member heteroaryl having from 1 to 3 nitrogen atoms in the ring, the carbons in the ring being optionally substituted with one or more groups, preferably one, two or three groups which are independently of each other halo, hydroxy, unsubstituted lower alkyl or unsubstituted lower alkoxy groups, 5-member heteroaryl having from 1 to 3 heteroatoms selected from the group consisting of nitrogen, oxygen and sulfur, the carbon and the nitrogen atoms in the group being optionally substituted with one or more groups, preferably one, two or three groups which are independently of each other halo, hydroxy, unsubstituted lower alkyl or unsubstituted lower alkoxy groups, 5- or 6-member heterocyclic group having from 1 to 3 heteroatoms selected from the group consisting of nitrogen, oxygen and sulfur, the carbon and nitrogen (if present) atoms in the group being optionally substituted with one or more groups, preferably one, two or three groups which are independently of each other halo, hydroxy, unsubstituted lower alkyl or unsubstituted lower alkoxy groups, mercapto, (unsubstituted lower alkyl)thio, arylthio optionally substituted with one or more groups, preferably one, two or three groups which are independently of each other halo, hydroxy, unsubstituted lower alkyl or alkoxy groups, cyano, acyl, thioacyl, O-carbamyl, N-carbamyl, O-thiocarbamyl, N-thiocarbamyl, C-amido, N-amido, nitro, N-sulfonamido, S-sulfonamido, RS(O)—, RS(O)₂—, —C(O)OR, RC(O)O—, and —NR₁₃R₁₄, wherein R₁₃ and R₁₄ are independently selected from the group consisting of hydrogen, unsubstituted lower alkyl, trihalomethyl, cycloalkyl, heterocyclic and aryl optionally substituted with one or more, groups, preferably one, two or three groups which are independently of each other halo, hydroxy, unsubstituted lower alkyl or unsubstituted lower alkoxy groups.

Preferably, the alkyl group is substituted with one or two substituents independently selected from the group consisting of hydroxy, 5- or 6-member heterocyclic group having from 1 to 3 heteroatoms selected from the group consisting of nitrogen, oxygen and sulfur, the carbon and nitrogen (if present) atoms in the group being optionally substituted with one or more groups, preferably one, two or three groups which are independently of each other halo, hydroxy, unsubstituted lower alkyl or unsubstituted lower alkoxy groups, 5-member heteroaryl having from 1 to 3 heteroatoms selected from the group consisting of nitrogen, oxygen and sulfur, the carbon and the nitrogen atoms in the group being optionally substituted with one or more groups, preferably one, two or three groups which are independently of each other halo, hydroxy, unsubstituted lower alkyl or unsubstituted lower alkoxy groups, 6-member heteroaryl having from 1 to 3 nitrogen atoms in the ring, the carbons in the ring being optionally substituted with one or more groups, preferably one, two or three groups which are independently of each other halo, hydroxy, unsubstituted lower alkyl or unsubstituted lower alkoxy groups, or —NR₁₃R₁₄, wherein R₁₃ and R₁₄ are independently selected from the group consisting of hydrogen and alkyl. Even more preferably the alkyl group is substituted with one or two substituents which are independently of each other hydroxy, dimethylamino, ethylamino, diethylamino, dipropylamino, pyrrolidino, piperidino, morpholino, piperazino, 4-lower alkylpiperazino, phenyl, imidazolyl, pyridinyl, pyridazinyl, pyrimidinyl, oxazolyl, triazinyl, and the like.

“Cycloalkyl” refers to a 3 to 8 member all-carbon monocyclic ring, an all-carbon 5-member/6-member or 6-member/6-member fused bicyclic ring or a multicyclic fused ring (a “fused” ring system means that each ring in the system shares an adjacent pair of carbon atoms with each other ring in the system) group wherein one or more of the rings may contain one or more double bonds but none of the rings has a completely conjugated pi-electron system.

Examples, without limitation, of cycloalkyl groups are cyclopropane, cyclobutane, cyclopentane, cyclopentene, cyclohexane, cyclohexadiene, adamantane, cycloheptane, cycloheptatriene, and the like. A cycloalkyl group may be substituted or unsubstituted. When substituted, the substituent group(s) is preferably one or more, more preferably one or two substituents, independently selected from the group consisting of unsubstituted lower alkyl, trihaloalkyl, halo, hydroxy, unsubstituted lower alkoxy, aryl optionally substituted with one or more, preferably one or two groups independently of each other halo, hydroxy, unsubstituted lower alkyl or unsubstituted lower alkoxy groups, aryloxy optionally substituted with one or more, preferably one or two groups independently of each other halo, hydroxy, unsubstituted lower alkyl or unsubstituted lower alkoxy groups, 6-member heteroaryl having from 1 to 3 nitrogen atoms in the ring, the carbons in the ring being optionally substituted with one or more, preferably one or two groups independently of each other halo, hydroxy, unsubstituted lower alkyl or unsubstituted lower alkoxy groups, 5-member heteroaryl having from 1 to 3 heteroatoms selected from the group consisting of nitrogen, oxygen and sulfur, the carbon and nitrogen atoms of the group being optionally substituted with one or more, preferably one or two groups independently of each other halo, hydroxy, unsubstituted lower alkyl or unsubstituted lower alkoxy groups, 5- or 6-member heterocyclic group having from 1 to 3 heteroatoms selected from the group consisting of nitrogen, oxygen and sulfur, the carbon and nitogen (if present) atoms in the group being optionally substituted with one or more, preferably one or two groups independently of each other halo, hydroxy, unsubstituted lower alkyl or unsubstituted lower alkoxy groups, mercapto, (unsubstituted lower alkyl)thio, arylthio optionally substituted with one or more, preferably one or two groups independently of each other halo, hydroxy, unsubstituted lower alkyl or unsubstituted lower alkoxy groups, cyano, acyl, thioacyl, O-carbamyl, N-carbamyl, O-thiocarbamyl, N-thiocarbamyl, C-amido, N-amido, nitro, N-sulfonamido, S-sulfonamido, RS(O)—, RS(O)₂—, —C(O)OR, RC(O)O—, and —NR₁₃R₁₄ are as defined above.

“Alkenyl” refers to a lower alkyl group, as defined herein, consisting of at least two carbon atoms and at least one carbon-carbon double bond. Representative examples include, but are not limited to, ethenyl, 1-propenyl, 2-propenyl, 1-, 2-, or 3-butenyl, and the like.

“Alkynyl” refers to a lower alkyl group, as defined herein, consisting of at least two carbon atoms and at least one carbon-carbon triple bond. Representative examples include, but are not limited to, ethynyl, 1-propynyl, 2-propynyl, 1-, 2-, or 3-butynyl, and the like.

“Aryl” refers to an all-carbon monocyclic or fused-ring polycyclic (i.e., rings which share adjacent pairs of carbon atoms) groups of 1 to 12 carbon atoms having a completely conjugated pi-electron system. Examples, without limitation, of aryl groups are phenyl, naphthalenyl and anthracenyl. The aryl group may be substituted or unsubstituted. When substituted, the substituted group(s) is preferably one or more, more preferably one, two or three, even more preferably one or two, independently selected from the group consisting of unsubstituted lower alkyl, trihaloalkyl, halo, hydroxy, unsubstituted lower alkoxy, mercapto, (unsubstituted lower alkyl)thio, cyano, acyl, thioacyl, O-carbamyl, N-carbamyl, O-thiocarbamyl, N-thiocarbamyl, C-amido, N-amido, nitro, N-sulfonamido, S-sulfonamido, RS(O)—, RS(O)₂—, —C(O)OR, RC(O)O—, and —NR₁₃R₁₄, with R₁₃ and R₁₄ as defined above. Preferably, the aryl group is optionally substituted with one or two substituents independently selected from halo, unsubstituted lower alkyl, trihaloalkyl, hydroxy, mercapto, cyano, N-amido, mono or dialkylamino, carboxy, or N-sulfonamyido.

“Heteroaryl” refers to a monocyclic or fused ring (i.e., rings which share an adjacent pair of atoms) group of 5 to 12 ring atoms containing one, two, or three ring heteroatoms selected from N, O, or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine and carbazole. The heteroaryl group may be substituted or unsubstituted. When substituted, the substituted group(s) is preferably one or more, more preferably one, two, or three, even more preferably one or two, independently selected from the group consisting of unsubstituted lower alkyl, trihaloalkyl, halo, hydroxy, unsubstituted lower alkoxy, mercapto, (unsubstituted lower alkyl)thio, cyano, acyl, thioacyl, O-carbamyl, N-carbamyl, O-thiocarbamyl, N-thiocarbamyl, C-amido, N-amido, nitro, N-sulfonamido, S-sulfonamido, RS(O)—, RS(O)₂—, —C(O)OR, RC(O)O—, and —NR₁₃R₁₄, with R₁₃ and R₁₄ as defined above. Preferably, the heteroaryl group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

“Heterocyclic” refers to a monocyclic or fused ring group having in the ring(s) of 5 to 9 ring atoms in which one or two ring atoms are heteroatoms selected from N, O, or S(O)ₙ (where n is an integer from 0 to 2), the remaining ring atoms being C. The rings may also have one or more double bonds. However, the rings do not have a completely conjugated pi-electron system. Examples, without limitation, of unsubstituted heterocyclic groups are pyrrolidino, piperidino, piperazino, morpholino, thiomorpholino, homopiperazino, and the like. The heterocyclic ring may be substituted or unsubstituted. When substituted, the substituted group(s) is preferably one or more, more preferably one, two or three, even more preferably one or two, independently selected from the group consisting of unsubstituted lower alkyl, trihaloalkyl, halo, hydroxy, unsubstituted lower alkoxy, mercapto, (unsubstituted lower alkyl)thio, cyano, acyl, thioacyl, O-carbamyl, N-carbamyl, O-thiocarbamyl, N-thiocarbamyl, C-amido, N-amido, nitro, N-sulfonamido, S-sulfonamido, RS(O)—, RS(O)₂—, —C(O)OR, RC(O)O—, and —NR₁₃R₁₄, with R₁₃ and R₁₄ as defined above. Preferably, the heterocyclic group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

Preferably, the heterocyclic group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

“Hydroxy” refers to an —OH group.

“Alkoxy” refers to both an —O-(unsubstituted alkyl) and an —O-(unsubstituted cycloalkyl) group. Representative examples include, but are not limited to, e.g., methoxy, ethoxy, propoxy, butoxy, cyclopropyloxy, cyclobutyloxy, cyclopentyloxy, cyclohexyloxy, and the like.

“Aryloxy” refers to both an —O-aryl and an —O-heteroaryl group, as defined herein. Representative examples include, but are not limited to, phenoxy, pyridinyloxy, furanyloxy, thienyloxy, pyrimidinyloxy, pyrazinyloxy, and the like, and derivatives thereof.

“Mercapto” refers to an —SH group.

“Alkylthio” refers to both an —S-(unsubstituted alkyl) and an —S-(unsubstituted cycloalkyl) group. Representative examples include, but are not limited to, e.g., methylthio, ethylthio, propylthio, butylthio, cyclopropylthio, cyclobutylthio, cyclopentylthio, cyclohexylthio, and the like.

“Arylthio” refers to both an —S-aryl and an —S-heteroaryl group, as defined herein. Representative examples include, but are not limited to, phenylthio, pyridinylthio, furanylthio, thientylthio, pyrimidinylthio, and the like and derivatives thereof.

“Acyl” refers to a —C(O)—R″ group, where R″ is selected from the group consisting of hydrogen, unsubstituted lower alkyl, trihalomethyl, unsubstituted cycloalkyl, aryl optionally substituted with one or more, preferably one, two, or three substituents selected from the group consisting of unsubstituted lower alkyl, trihalomethyl, unsubstituted lower alkoxy, halo and —NR₁₃R₁₄ groups, heteroaryl (bonded through a ring carbon) optionally substituted with one or more, preferably one, two, or three substituents selected from the group consisting of unsubstituted lower alkyl, trihaloalkyl, unsubstituted lower alkoxy, halo and —NR₁₃R₁₄ groups and heterocyclic (bonded through a ring carbon) optionally substituted with one or more, preferably one, two, or three substituents selected from the group consisting of unsubstituted lower alkyl, trihaloalkyl, unsubstituted lower alkoxy, halo and

—NR₁₃R₁₄ groups. Representative acyl groups include, but are not limited to, acetyl, trifluoroacetyl, benzoyl, and the like.

“Aldehyde” refers to an acyl group in which R″ is hydrogen.

“Thioacyl” refers to a —C(S)—R″ group, with R″ as defined herein.

“Ester” refers to a —C(O)O—R″ group with R″ as defined herein except that R″ cannot be hydrogen.

“Acetyl” group refers to a —C(O)CH₃ group.

“Halo” group refers to fluorine, chlorine, bromine or iodine, preferably fluorine or chlorine.

“Trihalomethyl” group refers to a —CX₃ group wherein X is a halo group as defined herein.

“Methylenedioxy” refers to a —OCH₂O— group where the two oxygen atoms are bonded to adjacent carbon atoms.

“Ethylenedioxy” group refers to a —OCH₂CH₂O— where the two oxygen atoms are bonded to adjacent carbon atoms.

“S-sulfonamido” refers to a —S(O)₂NR₁₃R₁₄ group, with R₁₃ and R₁₄ as defined herein.

“N-sulfonamido” refers to a —NR₁₃S(O)₂R group, with R₁₃ and R as defined herein.

“O-carbamyl” group refers to a —OC(O)NR₁₃R₁₄ group with R₁₃ and R₁₄ as defined herein.

“N-carbamyl” refers to an ROC(O)NR₁₄— group, with R and R₁₄ as defined herein.

“O-thiocarbamyl” refers to a —OC(S)NR₁₃R₁₄ group with R₁₃ and R₁₄ as defined herein.

“N-thiocarbamyl” refers to a ROC(S)NR₁₄— group, with R and R₁₄ as defined herein.

“Amino” refers to an —NR₁₃R₁₄ group, wherein R₁₃ and R₁₄ are both hydrogen.

“C-amido” refers to a —C(O)NR₁₃R₁₄ group with R₁₃ and R₁₄ as defined herein.

“N-amido” refers to a RC(O)NR₁₄— group, with R and R₁₄ as defined herein.

“Nitro” refers to a —NO₂ group.

“Haloalkyl” means an unsubstituted alkyl, preferably unsubstituted lower alkyl as defined above that is substituted with one or more same or different halo atoms, e.g., —CH₂Cl, —CF₃, —CH₂CF₃, —CH₂CCl₃, and the like.

“Aralkyl” means unsubstituted alkyl, preferably unsubstituted lower alkyl as defined above which is substituted with an aryl group as defined above, e.g., —CH₂phenyl, —(CH₂)₂phenyl, —(CH₂)₃phenyl, CH₃CH(CH₃)CH₂phenyl, and the like and derivatives thereof.

“Heteroaralkyl” group means unsubstituted alkyl, preferably unsubstituted lower alkyl as defined above which is substituted with a heteroaryl group, e.g.,

—CH₂pyridinyl, —(CH₂)₂pyrimidinyl, —(CH₂)₃imidazolyl, and the like, and derivatives thereof.

“Monoalkylamino” means a radical —NHR′ where R′ is an unsubstituted alkyl or unsubstituted cycloalkyl group as defined above, e.g., methylamino, (1-methylethyl)amino, cyclohexylamino, and the like.

“Dialkylamino” means a radical —NR′R′ where each R′ is independently an unsubstituted alkyl or unsubstituted cycloalkyl group as defined above, e.g., dimethylamino, diethylamino, (1-methylethyl)-ethylamino, cyclohexylmethylamino, cyclopentylmethylamino, and the like.

“Cyanoalkyl” means unsubstituted alkyl, preferably unsubstituted lower alkyl as defined above, which is substituted with 1 or 2 cyano groups.

“Optional” or “optionally” means that the subsequently described event or circumstance may but need not occur, and that the description includes instances where the event or circumstance occurs and instances in which it does not. For example, “heterocycle group optionally substituted with an alkyl group” means that the alkyl may but need not be present, and the description includes situations where the heterocycle group is substituted with an alkyl group and situations where the heterocyclo group is not substituted with the alkyl group.

A “pharmaceutical composition” refers to a mixture of one or more of the compounds described herein, or physiologically/pharmaceutically acceptable salts or prodrugs thereof, with other chemical components, such as physiologically/pharmaceutically acceptable carriers and excipients. The purpose of a pharmaceutical composition is to facilitate administration of a compound to an organism.

Prodrugs of a compound of Formula I or Formula II are within the scope of this invention. Additionally, a compound of Formula I or Formula II itself may act as a prodrug. A “prodrug” refers to an agent that is converted into a parent drug in vivo. Prodrugs are often useful because, in some situations, they may be easier to administer than the parent drug. They may, for instance, be bioavailable by oral administration whereas the parent drug is not. The prodrug may also have improved solubility in pharmaceutical compositions over the parent drug. An example, without limitation, of a prodrug would be a compound of the present invention that is administered as an ester (the “prodrug”) to facilitate transmittal across a cell membrane where water solubility is detrimental to mobility but then is metabolically hydrolyzed to the carboxylic acid, the active entity, once inside the cell where water solubility is beneficial.

A further example of a prodrug might be a short polypeptide, for example, without limitation, a 2-10 amino acid polypeptide, bonded through a terminal amino group to a carboxy group of a compound of this invention, wherein the polypeptide is hydrolyzed or metabolized in vivo to release the active molecule.

Additionally, it is contemplated that a compound of Formula I or Formula II would be metabolized by enzymes in the body of the organism such as human being to generate a metabolite that can modulate the activity of the protein kinases. Such metabolites are within the scope of the present invention.

As used herein, a “physiologically/pharmaceutically acceptable carrier” refers to a carrier or diluent that does not cause significant irritation to an organism and does not abrogate the biological activity and properties of the administered compound.

An “pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

As used herein, the term “pharmaceutically acceptable salt” refers to those salts that retain the biological effectiveness and properties of the parent compound. Such salts include:

(i) acid addition salt which is obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid or malonic acid and the like, preferably hydrochloric acid or (L)-malic acid such as the L-malate salt of 5-(5-fluoro-2-oxo-1,2-dihydroindol-3-ylidenemethyl)-2,4-dimethyl-1H-pyrrole-3-carboxylic acid(2-diethylaminoethyl)amide; or

(ii) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Method” refers to manners, means, techniques and procedures for accomplishing a given task including, but not limited to, those manners, means, techniques and procedures either known to, or readily developed from known manners, means, techniques and procedures by, practitioners of the chemical, pharmaceutical, biological, biochemical and medical arts.

“In vivo” refers to procedures performed within a living organism such as, without limitation, a mouse, rat or rabbit.

“Treat”, “treating” and “treatment” refer to a method of alleviating, ameliorating, abrogating or relieving a disease condition and/or any of its attendant symptoms.

“Patient” refers to any living entity comprised of at least one cell. A living organism can be as simple as, for example, a single eukaryotic cell or as complex as a mammal, including a human being.

“Therapeutically effective amount” refers to that amount of the compound being administered which will prevent, alleviate, ameliorate or relieve to some extent, one or more of the signs or symptoms of the disorder being treated.

“CSF1R” denotes the macrophage colony stimulating factor receptor, and includes what may be designated CSF-1 receptor, M-CSF receptor and/or c-fms gene products. Also included are any constitutive parts or elements of a macrophage colony stimulating factor receptor.

Osteolytic conditions that can be treated according to the present invention include the systemic condition known as osteoporosis. The osteoporosis may be attributed to (1) menopause in women, (2) aging in men or women, (3) suboptimal bone growth during childhood and adolescence that resulted in failure to reach peak bone mass, and/or (4) bone loss secondary to other disease conditions, eating disorders, medications and/or medical treatments. Another systemic condition that may be treated is Pagets disease, which comprises an excessive osteolytic component.

Other osteolytic diseases that can be treated according to the present invention are more localized. A particular example is metastatic tumor-induced osteolysis. In this condition, bone cancers or bone metastases induce localized osteolysis that causes pain, bone weakness and fractures. Such localized osteolysis also permits tumors to grow larger by creating more space for them in the bone and releasing growth factors from the bone matrix. Cancers presently known to cause tumor-induced osteolysis include hematological malignancies (e.g., myeloma and lymphoma) and solid tumors (e.g., breast, prostate, lung, renal and thyroid), all of which the present invention contemplates treating.

As noted above, the inventors have discovered that compounds of Formula I and Formula II inhibit phosphorylation of M-CSF receptors. Thus, the invention includes methods of inhibiting M-CSF receptor phosphorylation by administering a compound of Formula I or Formula II to a patient.

Additionally, the invention includes methods of treating cancers that express CSF1R. Examples of such cancers include, but are not limited to, breast cancers and cancers of the female reproductive tract such as ovarian cancer and endometrial cancer. Other cancers include myelodysplastic syndromes (MDS), acute myeloid leukemia (AML) and acute promyelocytic leukemia (APML). Therefore, the compounds of Formula I or Formula II may be administered to treat patients with M-CSF receptor positive cancers.

Prior to administering a compound of Formula I or Formula II to a cancer patient, the cancer may be tested to determine whether it expresses CSF1R. Such testing may directly detect CSF1R proteins (e.g., immunological assays such as ELISA, RIPA, IHC staining) or may do so indirectly (e.g., detecting gene transcripts via hybridization methods such as ISH). Such procedures are commonly known in the art, and have successfully been performed for CSF1R by Kacinski et al. (23, 26-27), Tang et al. (34), and Toy et al. (28). The fact that a cancer expresses CSF1R indicates that treatment with a compound of Formula I or Formula II will be useful.

### Administration and Pharmaceutical Composition

The claimed methods involve administration of a compound of Formula I or Formula II or a pharmaceutically acceptable salt thereof, to a human patient. Alternatively, the compounds of Formula I or Formula II can be administered in pharmaceutical compositions in which the foregoing materials are mixed with suitable carriers or excipient(s). Techniques for formulation and administration of drugs may be found in “Remington's Pharmacological Sciences,” Mack Publishing Co., Easton, Pa., latest edition.

As used herein, “administer” or “administration” refers to the delivery of a compound of Formula I or Formula II or a pharmaceutically acceptable salt thereof or of a pharmaceutical composition containing a compound of Formula I or Formula II or a pharmaceutically acceptable salt thereof of this invention to an organism for the purpose of treating excessive osteolysis or cancer.

Suitable routes of administration may include, without limitation, oral, rectal, transmucosal or intestinal administration or intramuscular, subcutaneous, intramedullary, intrathecal, direct intraventricular, intravenous, intravitreal, intraperitoneal, intranasal, or intraocular injections. The preferred routes of administration are oral and parenteral.

Furthermore, one may administer the drug in a targeted drug delivery system, for example, in a liposome coated with tumor-specific antibody. The liposomes will be targeted to and taken up selectively by the tumor or osteoclast progenitor.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethylcellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, or liquid polyethylene glycols. Stabilizers also may be added in these formulations.

Pharmaceutical compositions that may also be used include hard gelatin capsules. As a non-limiting example, compound 1 in a capsule oral drug product formulation may be as 50 and 200 mg dose strengths. The two dose strengths are made from the same granules by filling into different size hard gelatin capsules, size 3 for the 50 mg capsule and size 0 for the 200 mg capsule.

The capsules may be packaged into brown glass or plastic bottles to protect the active compound from light. The containers containing the active compound capsule formulation must be stored at controlled room temperature (15-30° C.).

For administration by inhalation, the compounds for use according to the present invention are conveniently delivered in the form of an aerosol spray using a pressurized pack or a nebulizer and a suitable propellant, e.g., without limitation, dichlorodifluoromethane, trichlorofluoromethane, dichlorotetra-fluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

The compounds may also be formulated for parenteral administration, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

Pharmaceutical compositions for parenteral administration include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

A non-limiting example of a pharmaceutical carrier for the hydrophobic compounds of the invention is a cosolvent system comprising benzyl alcohol, a nonpolar surfactant, a water-miscible organic polymer and an aqueous phase such as the VPD co-solvent system. VPD is a solution of 3% w/v benzyl alcohol, 8% w/v of the nonpolar surfactant Polysorbate 80, and 65% w/v polyethylene glycol 300, made up to volume in absolute ethanol. The VPD co-solvent system (VPD:D5W) consists of VPD diluted 1:1 with a 5% dextrose in water solution. This co-solvent system dissolves hydrophobic compounds well, and itself produces low toxicity upon systemic administration. Naturally, the proportions of such a co-solvent system may be varied considerably without destroying its solubility and toxicity characteristics. Furthermore, the identity of the co-solvent components may be varied: for example, other low-toxicity nonpolar surfactants may be used instead of Polysorbate 80, the fraction size of polyethylene glycol may be varied, other biocompatible polymers may replace polyethylene glycol, e.g., polyvinyl pyrrolidone, and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. In addition, certain organic solvents such as dimethylsulfoxide also may be employed, although often at the cost of greater toxicity.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions herein also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Examples of formulations for use in the present invention are in Tables 1-3 (below), which can be found in U.S. patent application Ser. No. 10/237,966, filed Sep. 10, 2002, now a provisional application, which is expressly incorporated in its entirety by reference.

TABLE 1
Composition of 5-(5-fluoro-2-oxo-1,2-dihydro-indol-3-ylidenemethyl)-
2,4-dimethyl-1H-pyrrole-3-carboxylic acid (2-diethylamino-ethyl)-
amide hard gelatin capsules

| | Concentration | Amount in 50 | Amount in 75 | Amount in 200 |
|---|---|---|---|---|
| | in Granulation | mg Capsule | mg Capsule | mg Capsule |
| Ingredient Name | (% w/w) | (mg) | (mg) | (mg) |
| API | 65.0 | 50.0 | 75.0 | 200.0 |
| Mannitol | 23.5 | 18.1 | 27.2 | 72.4 |
| Croscaramellose | 6.0 | 4.6 | 6.9 | 18.4 |
| Sodium^{e} | | | | |
| Povidone (K-25) | 5.0 | 3.8 | 5.7 | 15.2 |
| Magnesium | 0.5 | 0.38 | 0.57 | 1.52 |
| Stearate | | | | |
| Capsule | — | Size 1 | Size 3 | Size 0 |

TABLE 2
Composition of 5-(5-fluoro-2-oxo-1,2-dihydro-indol-3-
ylidenemethyl)-2,4-dimethyl-1H-pyrrole-3-carboxylic acid
(2-diethylamino-ethyl)-amide L-malate hard gelatin capsules

| | Concentration | |
|---|---|---|
| Ingredient | in Granulation | Amount in 50 mg |
| Name/Grade | (% w/w) | Capsule (mg) |
| API | 75.0 | 66.800^{c} |
| Mannitol | 13.5 | 12.024 |
| Croscaramellose | 6.0 | 5.344 |
| Sodium^{e} | | |
| Povidone (K-25) | 5.0 | 4.453 |
| Magnesium Stearate | 0.5 | 1.445 |
| Capsule | — | Size 3 |

TABLE 3
Composition of 5-(5-fluoro-2-oxo-1,2-dihydro-indol-3-ylidenemethyl)-
2,4-dimethyl-1H-pyrrole-3-carboxylic acid (2-diethylamino-ethyl)-
amide L-malate hard gelatin capsules

| | Concentration | Amount in 25 | Amount in 50 | Amount in 100 |
|---|---|---|---|---|
| Ingredient | in Granulation | mg Capsule | mg Capsule | mg Capsule |
| Name/Grade | (% w/w) | (mg) | (mg) | (mg) |
| API^{a} | 40.0 | 33.400^{d} | 66.800^{c} | 200.0^{b} |
| Mannitol | 47.5 | 39.663 | 79.326 | 158.652 |
| Croscaramellose | 6.0 | 5.010 | 10.020 | 20.04 |
| Sodium^{e} | | | | |
| Povidone (K-25) | 5.0 | 4.175 | 8.350 | 16.700 |
| Magnesium | 1.5 | 1.252 | 2.504 | 5.008 |
| Stearate | | | | |
| Capsule | — | Size 3 | Size 1 | Size 0 |

^{a}Drug substance quantity required for the batch will be adjusted to have 100% of labeled strength for capsules. Appropriate adjustment will be made to mannitol quantity to keep the same fill weight for each strength.
^{b}Quantity equivalent to 100 mg free base.
^{c}Quantity equivalent to 50 mg free base.
^{d}Quantity equivalent to 25 mg free base.
^{e}Half intragranular half extragranular.

Many of the compounds of Formula I and Formula II may be provided as physiologically acceptable salts wherein the compound may form the negatively or the positively charged species. Examples of salts in which the compound forms the positively charged moiety include, without limitation, quaternary ammonium, salts such as the hydrochloride, sulfate, carbonate, lactate, tartrate, malate, maleate, succinate wherein the nitrogen atom of the quaternary ammonium group is a nitrogen of the selected compound of this invention which has reacted with the appropriate acid. Salts in which a compound of this invention forms the negatively charged species include, without limitation, the sodium, potassium, calcium and magnesium salts formed by the reaction of a carboxylic acid group in the compound with an appropriate base (e.g. sodium hydroxide (NaOH), potassium hydroxide (KOH), Calcium hydroxide (Ca(OH)₂), etc.).

Pharmaceutical compositions suitable for use in the present invention include compositions wherein the active ingredients are contained in an amount sufficient to achieve the intended purpose, i.e., a therapeutically effective amount.

Determination of a therapeutically effective amount is well within the capability of those skilled in the art, especially in light of the detailed disclosure provided herein.

For any compound used in the methods of the invention, the therapeutically effective amount or dose can be estimated initially from cell culture assays. Then, the dosage can be formulated for use in animal models so as to achieve a circulating concentration range that includes the IC₅₀ as determined in cell culture (i.e., the concentration of the test compound which achieves a half-maximal inhibition of phosphorylation of CSF1R). Such information can then be used to more accurately determine useful doses in humans.

Toxicity and therapeutic efficacy of the compounds described herein can be determined by standard pharmaceutical procedures in cell cultures or experimental animals, e.g., by determining the IC₅₀ and the LD₅₀, wherein the LD₅₀ is the concentration of test compound which achieves a half-maximal inhibition of lethality, for a subject compound. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide plasma levels of the active species that are sufficient to maintain the kinase modulating effects. These plasma levels are referred to as minimal effective concentrations (MECs). The MEC will vary for each compound but can be estimated from in vitro data, e.g., the concentration necessary to achieve 50-90% inhibition of a kinase may be ascertained using the assays described herein. Dosages necessary to achieve the MEC will depend on individual characteristics and route of administration. HPLC assays or bioassays can be used to determine plasma concentrations.

Dosage intervals can also be determined using MEC value. Compounds should be administered using a regimen that maintains plasma levels above the MEC for 10-90% of the time, preferably between 30-90% and most preferably between 50-90%.

At present, the therapeutically effective amounts of compounds of Formula I or Formula II may range from approximately 25 mg/m2 to 1500 mg/m2 per day; preferably about 3 mg/m2/day. Even more preferably 50 mg/qm qd till 400 mg/qd.

In cases of local administration or selective uptake, the effective local concentration of the drug may not be related to plasma concentration and other procedures known in the art may be employed to determine the correct dosage amount and interval.

The amount of a composition administered will, of course, be dependent on the subject being treated, the severity of the affliction, the manner of administration, the judgment of the prescribing physician, etc.

It is contemplated that the inventive method could be used in combination with other therapies, including chemotherapies, radiation therapies and surgical therapies for cancer. For combination therapies and pharmaceutical compositions described herein, the effective amounts of the compound of the invention and of the other agent can be determined by those of ordinary skill in the art, based on the effective amounts for the compounds described herein and those known or described for the other agent. The formulations and route of administration for such therapies and composition can be based on the information described herein for compositions and therapies comprising the compound of the invention as the sole active agent and on information provided for the chemotherapeutic and other agent in combination therewith.

Specifically, it is contemplated that the described compounds may be combined with bisphosphonate or with hormonal therapy (e.g., aromatose inhibitors) to prevent bone breakdown in breast cancer. It is further contemplated that the compounds may be combined with all-trans retinoic acid (ATRA) in the treatment of AML and other cancers.

### General Synthetic Procedure

The following general methodology may be employed to prepare the compounds of this invention:

The appropriately substituted 2-oxindole (1 equiv.), the appropriately substituted aldehyde (1.2 equiv.) and a base (0.1 equiv.) are mixed in a solvent (1-2 ml/mmol 2-oxindole) and the mixture is then heated for from about 2 to about 12 hours. After cooling, the precipitate that forms is filtered, washed with cold ethanol or ether and vacuum dried to give the solid product. If no precipitate forms, the reaction mixture is concentrated and the residue is triturated with dichloromethane/ether, the resulting solid is collected by filtration and then dried. The product may optionally be further purified by chromatography.

The base may be an organic or an inorganic base. If an organic base is used, preferably it is a nitrogen base. Examples of organic nitrogen bases include, but are not limited to, diisopropylamine, trimethylamine, triethylamine, aniline, pyridine, 1,8-diazabicyclo[5.4.1]undec-7-ene, pyrrolidine and piperidine.

Examples of inorganic bases are, without limitation, ammonia, alkali metal or alkaline earth hydroxides, phosphates, carbonates, bicarbonates, bisulfates and amides. The alkali metals include, lithium, sodium and potassium while the alkaline earths include calcium, magnesium and barium.

In a presently preferred embodiment of this invention, when the solvent is a protic solvent, such as water or alcohol, the base is an alkali metal or an alkaline earth inorganic base, preferably, a alkali metal or an alkaline earth hydroxide.

It will be clear to those skilled in the art, based both on known general principles of organic synthesis and on the disclosures herein which base would be most appropriate for the reaction contemplated.

The solvent in which the reaction is carried out may be a protic or an aprotic solvent, preferably it is a protic solvent. A “protic solvent” is a solvent which has hydrogen atom(s) covalently bonded to oxygen or nitrogen atoms which renders the hydrogen atoms appreciably acidic and thus capable of being “shared” with a solute through hydrogen bonding. Examples of protic solvents include, without limitation, water and alcohols.

An “aprotic solvent” may be polar or non-polar but, in either case, does not contain acidic hydrogens and therefore is not capable of hydrogen bonding with solutes. Examples, without limitation, of non-polar aprotic solvents, are pentane, hexane, benzene, toluene, methylene chloride and carbon tetrachloride. Examples of polar aprotic solvents are chloroform, tetrahydro-furan, dimethylsulfoxide and dimethylformamide.

In a presently preferred embodiment of this invention, the solvent is a protic solvent, preferably water or an alcohol such as ethanol.

The reaction is carried out at temperatures greater than room temperature. The temperature is generally from about 30° C. to about 150° C., preferably about 80° C. to about 100° C., most preferable about 75° C. to about 85° C., which is about the boiling point of ethanol. By “about” is meant that the temperature range is preferably within 10 degrees Celsius of the indicated temperature, more preferably within 5 degrees Celsius of the indicated temperature and, most preferably, within 2 degrees Celsius of the indicated temperature. Thus, for example, by “about 75° C.” is meant 75° C.±10° C., preferably 75° C.±5° C. and most preferably, 75° C.±2° C.

2-Oxindoles and aldehydes, may be readily synthesized using techniques well known in the chemical arts. It will be appreciated by those skilled in the art that other synthetic pathways for forming the compounds of the invention are available and that the following is offered by way of example and not limitation.

Compounds of the present invention are prepared according to the following methodologies and as described, e.g., in U.S. patent application Ser. No. 09/783,264 and WO 01/60814, WO 00/08202, U.S. Provisional Application No. 60/312,353, filed Aug. 15, 2001, now U.S. patent application Ser. No. 10/281,985, filed Aug. 13, 2002, U.S. Provisional Application No. 60/411,732, filed Sep. 18, 2002, U.S. Provisional Application No. 60/328,226, filed Oct. 10, 2001, now U.S. patent application Ser. No. 10/268,082, filed Oct. 10, 2002 and U.S. patent application Ser. No. 10/076,140, filed Feb. 15, 2002, all of which are incorporated by reference in their entirety. Additionally, the disclosures of U.S. provisional applications No. 60/448,874 and No. 60/448,922, filed Feb. 24, 2003, are incorporated herein by reference.

### Synthetic Methodologies

### Method A: Formylation of Pyrroles

POCl₃ (1.1 equiv.) is added dropwise to dimethylformamide (3 equiv.) at −10° C. followed by addition of the appropriate pyrrole dissolved in dimethylformamide. After stirring for two hours, the reaction mixture is diluted with H₂O and basified to pH 11 with 10 N KOH. The precipitate which forms is collected by filtration, washed with H₂O and dried in a vacuum oven to give the desired aldehyde.

### Method B: Saponification of Pyrrolecarboxylic Acid Esters

A mixture of a pyrrolecarboxylic acid ester and KOH (2-4 equiv.) in EtOH is refluxed until reaction completion is indicated by thin layer chromatography (TLC). The cooled reaction mixture is acidified to pH 3 with 1 N HCl. The precipitate that forms is collected by filtration, washed with H₂O and dried in a vacuum oven to give the desired pyrrolecarboxylic acid.

### Method C: Amidation

To a stirred solution of a pyrrolecarboxylic acid dissolved in dimethylformamide (0.3M) is added 1-ethyl-3-(3-dimethylamino-propyl)carbodiimide (1.2 equiv.), 1-hydroxybenzotriazole (1.2 equiv.), and triethylamine (2 equiv.). The appropriate amine is added (1 equiv.) and the reaction stirred until completion is indicated by TLC. Ethyl acetate is then added to the reaction mixture and the solution washed with saturated NaHCO₃ and brine (with extra salt), dried over anhydrous MgSO₄ and concentrated to afford the desired amide.

### Method D: Condensation of Aldehydes and Oxindoles Containing Carboxylic Acid Substituents

A mixture of the oxindole (1 equivalent), 1 equivalent of the aldehyde and 1-3 equivalents of piperidine (or pyrrolidine) in ethanol (0.4 M) is stirred at 90-100° C. until reaction completion is indicated by TLC. The mixture is then concentrated and the residue acidified with 2N HCl. The precipitate that forms is washed with H₂O and EtOH and then dried in a vacuum oven to give the product.

### Method E: Condensation of Aldehydes and Oxindoles Not Containing Carboxylic Acid Substituents

A mixture of the oxindole (1 equivalent), 1 equivalent of the aldehyde and 1-3 equivalents of piperidine (or pyrrolidine) in ethanol (0.4 M) is stirred at 90-100° C. until reaction completion is indicated by TLC. The mixture is cooled to room temperature and the solid that forms is collected by vacuum filtration, washed with ethanol and dried to give the product. If a precipitate does not form upon cooling of the reaction mixture, the mixture is concentrated and purified by column chromatography.

The following examples are given to illustrate the present invention. It should be understood, however, that the invention is not to be limited to the specific conditions or details described in these examples. Throughout the specification, any and all references to publicly available documents are specifically incorporated into this patent application by reference.

### SYNTHETIC EXAMPLES

### Example 1

### Synthesis of (3Z)-3-{[3,5-dimethyl-4-(morpholin-4-yl)piperidin-1-ylcarbonyl]-1H-pyrrol-2-ylmethylidene}-5-fluoro-1,3-dihydro-2H-indol-2-one (Compound 9)

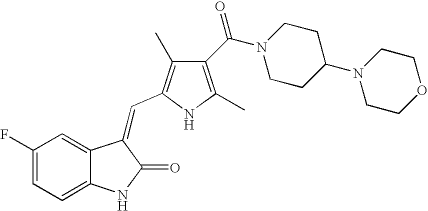

Step 1

To a stirred mixture of 4-amino-1-benzylpiperidine (Aldrich, 1.53 mL, 7.5 mmol), K₂CO₃ (2.28 g, 16.5 mmol), and DMF (15 mL) heated at 50° C. was added dropwise over 60 min bis(2-bromoethyl) ether (Aldrich, tech. 90%, 0.962 mL, 7.65 mmol). After stirring 6 h at 80° C., TLC (90:10:1 chloroform/MeOH/aq. conc. NH₄OH) indicated formation of a new spot. Heating was continued as the solvent was evaporated by blowing with a stream of nitrogen over 2 h. The crude material was relatively pure, but subjected to a relatively short silica gel column (1% to 6% gradient of 9:1 MeOH/aq. NH₄OH in chloroform). Evaporation of the pure fractions gave ˜1.7 g of the diamine 4-(morpholin-4-yl)-1-benzylpiperidine as a waxy solid.

¹HNMR (400 MHz, d₆-DMSO) δ 7.31 (m, 4H), 7.26 (m 1H), 3.72 (t, J=4.7 Hz, 4H), 3.49 (s, 2H), 2.94 (br d, J=5.9 Hz, 2H), 2.54 (t, J=4.7 Hz, 4H), 2.19 (tt, J=11.5, 3.9 Hz, 1H), 1.96 (td, J=11.7, 2.2 Hz, 2H), 1.78 (br d, J=12.5 Hz, 2H), 1.55 (m, 2H).

Step 2

A stirred mixture of Pd(OH)₂ (20% on carbon (<50% wet), 390 mg, 25 wt %), methanol (50 mL), and ≦1.7 M HCl (3 eq, ˜10.6 mL—including water added later when ppt was seen) under nitrogen was exchanged to 1 atm. hydrogen atmosphere by flushing (˜20 sec) using a balloon of nitrogen into the vessel and out through an oil bubbler. After 20 min. the reaction mixture under hydrogen was heated to 50° C. and 4-(morpholin-4-yl)-1-benzylpiperidine (1.56 g, 6.0 mmol) in methanol (8 mL) was added dropwise over 30 min. After 10 h, tlc indicated all starting amine was consumed to a more polar spot (ninhydrin active). The reaction mixture was then filtered through Celite and evaporated to yield the 4-(morpholin-4-yl)piperidine dihydrochloride as an off-white solid. This material was subjected to free-basing using excess basic resin (>16 g, Bio-Rad Laboratories, AG 1-X8, 20-50 mesh, hydroxide form, methanol washed two times) and a methanol mixture of the amine hydrochloride. After swirling with the resin for 30 min., the methanol solution was decanted and evaporated to yield 932 mg of 4-(morpholin-4-yl)piperidine free base as a waxy crystalline solid.

¹HNMR (400 MHz, d₆-DMSO) δ 3.53 (br s, 4H), 3.30 (v br s, 1H(+H₂O)), 2.92 (br d, J=11.7 Hz, 1H), 2.41 (s, 4H), 2.35 (˜obscd t, J=11.7 Hz, 2H), 2.12 (br t, 1H), 1.65 (br d, J=11.7 Hz, 2H), 1.18 (br q, J=10.9 Hz, 2H); LCMS−APCI m/z 171 [M+1]⁺.

Step 3

(3Z)-3-(3,5-Dimethyl-4-carboxy-1H-pyrrol-2-ylmethylidene)-5-fluoro-1,3-dihydro-2H-indol-2-one (120 mg, 0.40 mmol), prepared as described in PCT Publication No 01/60814, and BOP (221 mg, 0.50 mmol) were suspended in DMF (5 mL) with good stirring at room temperature and triethylamine (134 μL, 0.96 mmol) was added. After 10-15 min., to the homogeneous reaction mixture was added the 4-(morpholin-4-yl)piperidine (85 mg, 0.50 mmol) all at once. The reaction mixture was stirred for 48 h (might be done much earlier), then transferred to a funnel containing chloroform-isopropanol (5/1) and 5% aq. LiCl. The cloudy-orange organic phase was separated, washed with additional 5% aq LiCl (2×), 1 M aq NaOH (3×), satd aq NaCl (1×), and then dried (Na₂SO₄) and evaporated to yield the crude product (96.3% pure; trace HMPA by ¹HNMR). This crude product was then further purified by passage through a very short column (3 cm) of silica gel (5 to 15% gradient of MeOH in DCM) where a trace of faster moving 3E-isomer was removed. The pure fractions were evaporated and recrystallized overnight from a satd EtOAc soln which was diluted with Et₂O (˜3-fold) and chilled at 0° C. The mother liquor was decanted to yield after full vacuum the desired compound as orange crystals (153 mg 85%).

¹HNMR (400 MHz, d₆-DMSO) δ 13.60 (s, 1H), 10.87 (s, 1H), 7.72 (dd, J=9.4, 2.7 Hz, 1H), 7.68 (s, 1H), 6.91 (td, J=9.3, 2.6 Hz, 1H), 6.82 (dd, J=8.6, 4.7 Hz, 1H), 3.54 (app br t, J=4.3 Hz, 4H), 3.31 (2×s, 3H+3H), 2.43 (br s, 4H), 2.36 (m, 1H), 2.25 (br m, 6H), 1.79 (br s, 2H), 1.22 (br s, 2H); LCMS m/z 453 [M+1]⁺.

Proceeding as described in Example 1 above but substituting (3Z)-3-(3,5-dimethyl-4-carboxy-1H-pyrrol-2-ylmethylidene)-5-fluoro-1,3-dihydro-2H-indol-2-one for (3Z)-3-(3,5-dimethyl-4-carboxy-1H-pyrrol-2-ylmethylidene)-1,3-dihydro-2H-indol-2-one gave (3Z)-3-{[3,5-dimethyl-4-(morpholin-4-yl)piperidin-1-ylcarbonyl]-1H-pyrrol-2-ylmethylidene}-,3-dihydro-2H-indol-2-one. ¹HNMR (400 MHz, d₆-DMSO) δ 13.55 (s, 1H), 10.87 (s, 1H), 7.74 (d, J=7.6 Hz, 1H), 7.59 (s, 1H), 7.11 (t, J=7.6 Hz, 1H), 6.97 (t, J=7.6 Hz, 1H), 6.86 (d, J=7.4 Hz, 1H), 3.54 (app br t, J=4.3 Hz, 4H), 3.31 (2×s, 3H+3H), 2.43 (br s, 4H), 2.35 (m, 1H), 2.28 (br m, 6H), 1.79 (br s, 2H), 1.22 (br s, 2H); LCMS m/z 435 [M+1]⁺.

Proceeding as described in Example 1 above but substituting (3Z)-3-(3,5-dimethyl-4-carboxy-1H-pyrrol-2-ylmethylidene)-5-fluoro-1,3-dihydro-2H-indol-2-one for (3Z)-3-(3,5-dimethyl-4-carboxy-1H-pyrrol-2-ylmethylidene)-5-chloro-1,3-dihydro-2H-indol-2-one gave (3Z)-3-{[3,5-dimethyl-4-(morpholin-4-yl)piperidin-1-ylcarbonyl]-1H-pyrrol-2-ylmethylidene}-5-chloro-1,3-dihydro-2H-indol-2-one.

¹HNMR (400 MHz, d₆-DMSO) δ 13.56 (s, 1H), 10.97 (s, 1H), 7.95 (d, J=2.0 Hz, 1H), 7.74 (s, 1H), 7.11 (dd, J=8.2, 2.0 Hz, 1H), 6.85 (d, J=8.2 Hz, 1H), 3.54 (app br t, J=4 Hz, 4H), 3.31 (2×s, 3H+3H), 2.43 (br s, 4H), 2.37 (m, 1H), 2.25 (br m, 6H), 1.79 (br s, 2H), 1.23 (br s, 2H); LCMS m/z 470 [M+1]⁺.

Proceeding as described in Example 1 above but substituting 4-(morpholin-4-yl)-piperidine with commercially available 4-(1-pyrrolidinyl)-piperidine gave (3Z)-3-{[3,5-dimethyl-4-[4-(pyrrolidin-1-yl)piperidin-1-ylcarbonyl]-1H-pyrrol-2-yl)methylidene]-5-fluoro-1,3-dihydro-2H-indol-2-one.

¹HNMR (400 MHz, d₆-DMSO) 6 E/Z isomer mixture; LCMS m/z 437 [M+1]⁺.

Synthesis of the above examples can proceed according to the procedure of U.S. Provisional Application No. 60/328,226, filed Oct. 10, 2001 and U.S. patent application Ser. No. 10/268,082, filed Oct. 10, 2002, incorporated by reference in its entirety.

### Example 2

### Synthesis of (3Z)-3-{[3,5-dimethyl-4-(morpholin-4-yl)azetidin-1-ylcarbonyl]-1H-pyrrol-2-ylmethylidene}-5-fluoro-1,3-dihydro-2H-indol-2-one

Step 1

A solution of 1-azabicyclo[1.1.0]butane, prepared from 2,3-dibromopropylamine hydrobromide (58.8 mmol) according to a known procedure described in Tetrahedron Letters 40 (1999) 3761-64, was slowly added to a solution of morpholine (15.7 ml; 180 mmol) and sulfuric acid (3.3 g of 96% soln.) in anhydrous non-denaturated ethanol (250 ml) at 0° C. The reaction mixture was stirred on ice bath for 30 min., then at room temperature for 8 h. Calcium hydroxide (5.5 g) and 100 ml of water was added and the obtained slurry was stirred for 1 h and then filtered through a pad of cellite. The filtrate was concentrated and distilled at reduced pressure (20 mm Hg) to remove water and an excess of morpholine. The distillation residue was re-distilled at high vacuum using a Kugelrohr apparatus to obtain a pure 4-(azetidin-3-yl)morpholine in 33% yield (2.759 g) as a colorless oily liquid.

¹³C-NMR (CDCl₃, 100 MHz): 66.71(2C), 59.37 (IC), 51.46 (2C), 49.95(2C) ¹H (CDCl₃, 400 MHz): 3.727 (t, J=4.4 Hz, 4H), 3.619 (t, J=8 Hz, 2H), 3.566 (t, J=8 Hz, 2H), 3.227 (m, J=7 Hz, 1H), 2.895 (br s, 1H), 2.329 (br s, 4H)

Step 2

1-(8-Azabenztriazolyl)-ester of (3Z)-3-({3,5-dimethyl-4-carboxy]1-H-pyrrol-2-yl}methylene)-5-fluoro-1.3-dihydro-2H-indol-2-one (0.5 mmol, 210 mg) [prepared by activating (3Z)-3-(3,3-dimethyl-4-carboxy-1-H-pyrrol-2-ylmethylene)-5-fluoro-1.3-dihydro-2H-indol-2-one (480 mg; 1.6 mmol) with the HATU reagent (570 mg, 1.5 mmol) in the presence of Hunig base (3.0 mmol, 0.525 ml) in DMF (5 ml) and isolated in pure form by precipitation with chloroform (5 ml) and drying on high vacuum in 92% yield (579 mg)] was suspended in anhydrous DMA (1.0 ml). A solution of 4-(azetidin-3-yl)-morpholine; (142.5 mg, 1 mmol) in anhydrous DMA (1.0 ml) was added in one portion and the obtained solution was stirred at room temperature for 20 min. The reaction mixture was evaporated at room temperature using an oil pump, the thick residue was diluted with 6 ml of a mixture of methanol plus diethyl amine (20:1; v/v), inoculated mechanically and placed into a refrigerator (+3° C.) for 8 hours. The precipitates were filtered (with a brief wash with an ice-cold methanol) and dried on high vacuum to give the desired product. 71.5% yield (152 mg of an orange solid)

LC/MS: +APCI: M+1=425; −APCI: M+1=423

¹⁹F-NMR (d-DMSO, 376.5 MHz): −122.94 (m, 1F)

¹H (d-DMSO, 400 MHz): 13.651 (s, 1H), 10.907 (s, 1H), 7.754 (dd, J=9.4 Hz, J=2.4 Hz, 1H), 7.700 (s, 1H), 6.935 (dt, J=8.2 Hz, J=2.4 Hz, 1H), 6.841 (dd, J=8.6 Hz, J=3.9 Hz; 1H), 3.963 (br s, 2H), 3.793 (br s, 2H), 3.581 (br t, J=4.3 Hz, 4H), 3.133 (m, 1H), 2.367 (s, 3H), 2.340 (s, 3H), 2.295 (br s, 4H)

Proceeding as described in Example 2 above but substituting (3Z)-3-(3,5-dimethyl-4-carboxy-1H-pyrrol-2-ylmethylidene)-5-fluoro-1,3-dihydro-2H-indol-2-one with (3Z)-3-(3,5-dimethyl-4-carboxy-1H-pyrrol-2-ylmethylidene)-5-chloro-1,3-dihydro-2H-indol-2-one gave (3Z)-3-{[3,5-dimethyl-4-(morpholin-4-yl)azetidin-1-ylcarbonyl]-1H-pyrrol-2-ylmethylidene}-5-chloro-1,3-dihydro-2H-indol-2-one as an orange solid.

LC/MS: +APCI: M+1=441; −APCI: M+1=440,441

¹H (d-DMSO, 400 MHz): 13.607 (s, 1H), 11.006 (s, 1H), 7.976 (d, J=2.0 Hz, 1H), 7.756 (s, 1H), 7.136 (dd, J=8.2 Hz, J=2.0 Hz, 1H), 6.869 (d, J=8.2 Hz, 1H), 3.964 (br s, 2H), 3.793 (br s, 2H), 3.582 (br t, J=4.3 Hz, 4H), 3.134 (m, 1H), 2.369 (s, 3H), 2.347 (s, 3H), 2.296 (br s, 4H)

Proceeding as described in Example 2 above but substituting 4-(azetidin-3-yl)morpholine with 4-(azetidin-3-yl)-cis-3,5-dimethylmorpholine (prepared in a procedure analogous to the preparation of 4-(azetidin-3-yl)-morpholine but using cis-3,5-dimethylmorpholine (20.7 g; 180 mmol) in place of morpholine) gave (3Z)-3-{[3,5-dimethyl-4-(2,5-dimethylmorpholin-4-yl)azetidin-1-ylcarbonyl]-1H-pyrrol-2-ylmethylidene}-5-fluoro-1,3-dihydro-2H-indol-2-one as an orange solid

LC/MS: +APCI: M+1=453; −APCI: M+1=451

¹⁹F-NMR (d-DMSO, 376.5 MHz): −122.94 (m, 1F)

¹H (d-DMSO, 400 MHz): 13.651 (s, 1H), 10.907 (s; 1H), 7.758 (dd, J=9.4 Hz, J=2.3 Hz; 1H), 7.700 (s, 1H), 6.935 (dt, J=8.6 Hz, J=2.7 Hz, 1H), 6.842 (dd, J=8.2 Hz, J=4.3 Hz, 1H), 3.961 (br s, 2H), 3.790 (br s, 2H), 3.546 (br m, 2H), 3.092 (m, 1H), 2.690 (br s; 2H), 2.364 (s, 3H), 2.338 (s, 3H), 1.492 (br m, 2H), 1.038 (br s, 6H)

Proceeding as described in Example 2 above but substituting (3Z)-3-(3,5-dimethyl-4-carboxy-1H-pyrrol-2-ylmethylidene)-5-fluoro-1,3-dihydro-2H-indol-2-one with (3Z)-3-(3,5-dimethyl-4-carboxy-1H-pyrrol-2-ylmethylidene)-5-chloro-1,3-dihydro-2H-indol-2-one and 4-(azetidin-3-yl)morpholine with 4-(azetidin-3-yl)-cis-3,5-dimethylmorpholine gave (3Z)-3-{[3,5-dimethyl-4-(3,5-dimethylmorpholin-4-yl)azetidin-1-ylcarbonyl]-1H-pyrrol-2-ylmethylidene}-5-chloro-1,3-dihydro-2H-indol-2-one as an orange solid.

LC/MS: +APCI: M+1=469, 470; −APCI: M+1=468,469

¹H (d-DMSO, 400 MHz): 13.606 (s, 1H), 11.008 (s, 1H), 7.979 (d, J=2.0 Hz, 1H), 7.758 (s, 1H), 7.138 (dd, J=8.2 Hz, J=2.0 Hz, 1H), 6.870 (d, J=8.2 Hz, 1H), 3.964 (br s, 2H), 3.790 (br s, 2H), 3.547 (br m, 2H), 3.095 (m, 1H), 2.691 (br s, 2H), 2.366 (s, 3H), 2.345 (s, 3H), 1.494 (br m, 2H), 1.039 (br s, 6H)

Proceeding as described in Example 1 above, but substituting 4-(morpholin-4-yl)-piperidine with 2-(R)-pyrrolidin-1-ylmethylpyrrolidine prepared as described below provided (3Z)-3-{[3,5-dimethyl-2R-(pyrrolidin-1-ylmethyl)pyrrolidin-1-ylcarbonyl]-1H-pyrrol-2-ylmethylidene}-5-fluoro-1,3-dihydro-2H-indol-2-one

Synthesis of 2(R)-pyrrolidin-1-ylmethylpyrrolidine

Step 1

To a solution of (+)-Carbobenzyloxy-D-proline (1.5 g, 6.0 mmol), EDC (2.3 g, 12.0 mmol) and HOBt (800 mg, 12.9 mmol) in DMF (20 ml) was added trietylamine (1.5 ml) and pyrrolidine (1.0 ml, 12.0 mmol). It was stirred for 18 h at rt. Sat. NaHCO₃ was added, it was extracted with CH2Cl2 (three times). The organic layers were separated and dried over Na₂SO₄. The solvent was removed and the residue was purified by silica gel chromatography (EtOAc) to give 1-(R)-[N-(benzyloxycarbonyl)-pyrolyl]pyrrolidine as a white solid (94%).

¹H NMR (400 MHz, CDCl₃, all rotamers) □ 1.57-1.66 (m, 1H), 1.71-2.02 (m, 5H), 2.04-2.19 (m, 2H), 3.26-3.43 (m, 3H), 3.44-3.78 (m, 3H), 4.41 (dd, J=4.5, 7.6 Hz, 0.5H), 4.52 (dd, J=3.7, 7.6 Hz, 0.5H), 4.99 (d, J=12.1 Hz, 0.5H), 5.05 (d, J=12.5 Hz, 0.5H), 5.13 (d, J=12.1 Hz, 0.5H), 5.20 (d, J=12.5 Hz, 0.5H), 7.27-7.38 (m, 5H).

Step 2

A mixture of 1-(R)-[N-(benzyloxycarbonyl)prolyl]pyrrolidine (2.7 g, 8.9 mmol) and 5% Pd-C catalyst (270 mg) in methanol (15 ml) were stirred under a hydrogen atmosphere for 20 h. The reaction mixture was filtered through celite and the solvent was removed yielding 2(R)-prolylpyrrolidine as a viscous oil (80%), which was used without further purification for the next step.

¹H NMR (400 MHz, d₆-DMSO) δ 1.52-1.78 (m, 5H), 1.82-1.89 (m, 2H), 1.97-2.04 (m, 1H), 2.63-2.71 (m, 1H), 2.97-3.02 (m, 1H), 3.22-3.35 (m, 3H), 3.48-3.54 (m, 1H), 3.72 (dd, J=6.1, 8.0 Hz, 1H).

Step 3

2-(R)-Prolylpyrrolidine (1.2 g, 7.1 mmol) was dissolved in THF (10 ml). The reaction mixture was cooled to ₀° C. and BH₃, 1M in THF (10 ml, 10 mmol) was dropwise at 0 C. The reaction mixture was refluxed for 16 h, 3 M HCl (4.7 ml). 2 M NaOH solution was added until pH 10 was reached. The product was extracted with 5% MeOH in CH₂Cl₂ (three times). The organic layers were dried over Na₂SO₄ and the solvent was removed to provide the title compound as a slightly yellow liquid (73%), which was used without further purification for the next step.

¹H NMR (400 MHz, d₆-DMSO) δ 1.22-1.30 (m, 1H), 1.55-1.69 (m, 6H), 1.71-1.79 (m, 1H), 2.26-2.30 (m, 1H), 2.33-2.38 (m, 1H), 2.40-2.45 (m, 4H), 2.65-2.71 (m, 1H), 2.78-2.84 (m, 1H), 3.02-3.09 (m, 1H).

Proceeding as described in Example 1 above, but substituting 4-(morpholin-4-yl)-piperidine with 2-(S)-pyrrolidin-1-ylmethylpyrrolidine (prepared as described above, by substituting (+)-carbobenzyloxy-D-proline with carbobenzyloxy-L-proline) provided (3 Z)-3-{[3,5-dimethyl-2S-(pyrrolidin-1-ylmethyl)pyrrolidin-1-ylcarbonyl]-1H-pyrrol-2-ylmethylidene}-5-fluoro-1,3-dihydro-2H-indol-2-one.

### Example 3

### Synthesis of 5-[5-fluoro-2-oxo-1,2-dihydro-indol-(3Z)-ylidene-methyl]-2,4-dimethyl-1H-pyrrole-3-carboxylic acid

Step 1

Dimethylformamide (25 mL, 3 eq.) was cooled with stirring in an ice bath. To this was added POCl₃ (1.1 eq., 10.8 mL). After 30 minutes, a solution of the 3,5-dimethyl-4-ethylester pyrrole (17.7 g, 105.8 mmol) in DMF (2M, 40 mL) was added to the reaction and stirring continued. After 2 hour, the reaction was diluted with water (250 mL) and basified to pH=11 with 1N aqueous NaOH. The white solid was removed by filtration, rinsing with water and then hexanes and dried to afford 5-formyl-2,4-dimethyl-1H-pyrrole-3-carboxylic acid ethyl ester (19.75 g, 95%) as a tan solid.

¹H NMR (360 MHz, DMSO-d₆) δ 12.11 (br s, 1H, NH), 9.59 (s, 1H, CHO), 4.17 (q, J=6.7 Hz, 2H, OCH₂CH₃), 2.44 (s, 3H, CH₃), 2.40 (s, 3H, CH₃), 1.26 (d, J=6.7 Hz, 3H, OCH₂CH₃).

Step 2

5-Formyl-2,4-dimethyl-1H-pyrrole-3-carboxylic acid ethyl ester (2 g, 10 mmol) was added to a solution of potassium hydroxide (3 g, 53 mmol) dissolved in methanol (3 mL) and water (10 mL). The mixture was refluxed for 3 hours, cooled to room temperature and acidified with 6 N hydrochloric acid to pH 3. The solid was collected by filtration, washed with water and dried in a vacuum oven overnight to give 5-formyl-2,4-dimethyl-1H-pyrrole-3-carboxylic acid (1.6 g, 93%).

¹H NMR (300 MHz, DMSO-d₆) δ 12.09 (s, br, 2H, NH & COOH), 9.59 (s, 1H, CHO), 2.44 (s, 3H, CH₃), 2.40 (s, 3H, CH₃).

Step 3

5-Fluoroisatin (8.2 g, 49.7 mmol) was dissolved in 50 mL of hydrazine hydrate and refluxed for 1 hour. The reaction mixtures were then poured in ice water. The precipitate was then filtered, washed with water and dried under vacuum oven to give 5-fluoro-2-oxindole (7.5 g).

Step 4

The reaction mixture of 5-fluorooxindole (100 mg, 0.66 mmol), 5-formyl-2,4-dimethyl-1H-pyrrole-3-carboxylic acid (133 mg, 0.79 mmol), and 10 drops of piperidine in ethanol (3 mL) was stirred at 60° C. overnight and filtered. The solid was washed with 1 M of aqueous hydrochloride solution, water, and dried to afford 5-(5-fluoro-2-oxo-1,2-dihydro-indol-3-ylidenemethyl)-2,4-dimethyl-1H-pyrrole-3-carboxylic acid (201 mg, quantitative) as a yellow solid. MS m/z (relative intensity, %) 299 ([M+1]+, 100).

### Example 4

### Synthesis of 5-(5-Fluoro-2-oxo-1,2-dihydro-indol-3-ylidene-methyl)-2,4-dimethyl-1H-pyrrole-3-carboxylic acid (3-diethylamino-2-hydroxy-propyl)-amide

Step 1

To 2-chloromethyloxirane (95 g, 1.03 mole) was added a mixture of water (3.08 g, 0.17 mole) and diethylamine (106.2 mL, 1.03 mole) at 30° C. The reaction mixture was then stirred at 28-35° C. for 6 hour and cooled to 20-25° C. to give 1-chloro-3-diethylamino-propan-2-ol.

Step 2

A solution of sodium hydroxide (47.9 g, 1.2 mole) in 78 mL water was added 1-chloro-3-diethylamino-propan-2-ol. The resultant was stirred at 20-25° C. for 1 hour, diluted with 178 mL of water and extracted with ether twice. The combined ether solution was dried with solid potassium hydroxide and evaporated to give 135 g of crude product which was purified by fraction distillation to give pure glycidyldiethylamine (98 g, 76%) as an oil.

Step 3

To the ice-cold solution of ammonium hydroxide (25 mL, 159 mmole) of 25% (w/w) was added glycidyldiethylamine dropwise (3.2 g, 24.8 mmol) over 10 minutes. The reaction mixture was stirred at 0-5° C. for 1 hour and then room temperature for 14 hours. The resulting reaction mixture was evaporated and distilled (84-90° C. at 500-600 mT) to yield 1-amino-3-diethylamino-propan-2-ol (3.3 g, 92%). MS m/z 147 ([M+1]⁺).

Step 4

To the solution of 5-formyl-2,4-dimethyl-1H-pyrrole-3-carboxylic acid (100 mg, 0.43 mmol), EDC (122.7 mg, 0.64 mmol) and HOBt (86.5 mg, 0.64 mmol) in 1.0 mL of DMF was added 1-amino-3-diethylamino-propan-2-ol (93.2 mg, 0.64 mmol). The resulting reaction solution was stirred at room temperature overnight and evaporated. The residue was suspended in 10 mL of water and filtered. The solid was washed with saturated sodium bicarbonate and water and dried in a high vacuum oven overnight to give crude product which was purified on column chromatography eluting with 6% methanol-dichlormethane containing triethylamine (2 drops/100 mL of 6% methanol-dichloromethane) to give 5-(5-fluoro-2-oxo-1,2-dihydro-indol-3-ylidenemethyl)-2,4-dimethyl-1H-pyrrole-3-carboxylic acid (3-diethylamino-2-hydroxy-propyl)-amide (62 mg, 34%) as a yellow solid.

¹H NMR (400 MHz, DMSO-d₆) δ 13.70 (s, 1H, NH-1), 10.90 (s, 1H, NH-1), 7.76 (dd, J=2.38, 9.33 Hz, 1H, H-4), 7.72 (s, 1H, vinyl-H), 7.60 (m, br., 1H, CONHCH₂CH(OH)—CH₂N(C₂H₅)₂-4′), 6.93 (dt, J=2.38, 8.99 Hz, 1H, H-5), 6.85 (dd, J=4.55, 8.99 Hz, 1H, H-6), 3.83 (m, br, 1H, OH), 3.33 (m, 4H), 2.67 (m, br, 5H), 2.46 (s, 3H, CH₃), 2.44 (s, 3H, CH₃), 1.04 (m, br, 6H, CH₃×2). MS m/z (relative intensity, %) 427 ([M+1]⁺, 100).

### Example 5

### Synthesis of 5-[5-Fluoro-2-oxo-1,2-dihydro-indol-(3Z)-ylidene-methyl]-2,4-dimethyl-1H-pyrrole-3-carboxylic acid (2-hydroxy-3-morpholin-4-yl-propyl)-amide (R), (S) and (R/S) (Compounds 4, 5 and 6)

Step 1

A mixture of morpholine (2.6 mL, 30 mmol) and epichlorohydrin (2.35 ml, 30 mmol) in ethanol (50 mL) was stirred at 70° C. overnight. After removing the solvent, the residue was diluted with methylene chloride (50 mL). The clear solid precipitated was collected by vacuum filtration to give 1-chloro-3-morpholin-4-yl-propan-2-ol (2.0 g, 37%). ¹H NMR (DMSO-d₆) δ 3.49 (t, J=4.8 Hz, 2H), 3.60 (t, J=4.6 Hz, 2H), 3.75 (m, 4H, 2×CH₂), 4.20 (dd, J=5.2, 12 Hz, 2H), 4.54 (m, 2H), 4.62 (m, 1H, CH), 6.64 (d, J=6.4 Hz, 1H, OH). MS (m/z) 180.2 (M+1).

Step 2

1-Chloro-3-morpholin-4-yl-propan-2-ol (2.0 g, 11 mmol) was treated with the solution of NH₃ in methanol (25% by weight, 20 mL) at room temperature. Nitrogen was bubbled into the reaction mixture to remove the ammonia. Evaporation of solvent gave the hydrogen chloride salt of 1-amino-3-morpholin-4-yl-propan-2-ol (2.0 g, 91%). ¹H NMR (DMSO-d₆) δ 2.30 (d, J=6.0 Hz, 2H), 2.36 (m, 4H, NCH₂), 2.65 (dd, J=8.4, 12.8 Hz, 1H), 2.91 (dd, J=3.6, 12.8 Hz, 1H), 3.52 (m, 4H, OCH₂), 3.87 (m, 1H, CH), 5.32 (s, 1H, OH), 8.02 (brs., 3H, NH₃⁺). MS (m/z) 161.1 (M+1).

Step 3

5-(5-Fluoro-2-oxo-1,2-dihydro-indol-3-ylidenemethyl)-2,4-dimethyl-1H-pyrrole-3-carboxylic acid (120 mg, 0.4 mmol) was condensed with 1-amino-3-morpholin-4-yl-propan-2-ol (74 mg, 0.48 mmol) to precipitate 5-[5-fluoro-2-oxo-1,2-dihydro-indol-(3Z)-ylidenemethyl]-2,4-dimethyl-1H-pyrrole-3-carboxylic acid (2-hydroxy-3-morpholin-4-yl-propyl)-amide (65 mg, 36%). The mother liquid was evaporated to dryness and the residue was purified by flash chromatography to give additional 2N (70 mg, 39%). ¹H NMR (DMSO-d₆) δ 2.28 (m, 1H), 2.32 (m, 1H), 2.40 (m, 4H), 2.40, 2.42 (2×s, 6H, 2×CH₃), 3.15 (s, 1H), 3.31 (m, 1H), 3.55 (m, 4H), 3.78 (m, 1H), 4.73 (brs, 1H, OH), 6.82 (dd, J=4.5, 8.4 Hz, 1H), 6.90 (td, ²J=2.8, ³J=10.0 Hz, 1H), 7.53 (m, 1H), 7.70 (s, 1H), 7.74 (dd, J=2.0, 9.6 Hz, 1H) (aromatic and vinyl), 10.87 (s, 1H, CONH), 13.66 (s, 1H, NH). LC-MS (m/z) 441.4 (M−1).

### Synthesis of 2-hydroxy-7-oxa-4-azoniaspiro[3,5]nonane chloride

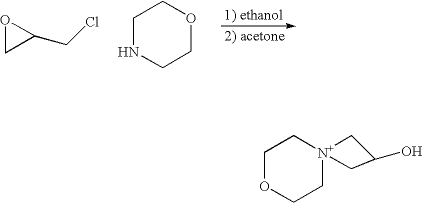

To a 1L 3-neck round bottom flask, fitted with a thermocouple, nitrogen inlet and a 250 ml addition funnel, was charged morpholine (91.5 g, 91.5 ml, 1.05 mole, 1.0 eq.) and 100 ml of ethanol. The solution was stirred rapidly while adding epichlorohydrin (10 g, 84.5 ml, 1.08 mole, 1.03 eq.) from the addition funnel over about 30 minutes. The temperature was monitored and when the pot temperature reached 27° C., the reaction was cooled with an ice water bath. The clear solution was stirred for 18 hours. The reaction was assayed by GC (dilute 5 drops of reaction mixture into 1 ml of ethanol and inject onto a 15 m DB-5 capillary GC column with the following run parameters, Injector 250° C., detector 250° C., initial oven temperature 28° C. warming to 250° C. at 10° C. per minute.) The reaction was complete with less than 3% morpholine remaining. The reaction was concentrated on the rotoevaporated at 50° C. with full house vacuum until no more distillate could be condensed. The resulting oil was stored at room temperature for 24-48 hours or until a significant mass of crystals was observed (seeded will speed up the process). The slurry was diluted with 250 ml of acetone and filtered. The solids were dried in the vacuum oven at 60° C. for 18-24 hours. This provided 84 g of crystalline product. The mother liquors could be concentrated and the crystallization process repeated in increase recovery. ¹H NMR (400 MHz, DMSO-d₆) δ 6.55 (d, 1H), 4.64 (m, 1H), 4.53 (m, 2H), 4.18 (m, 2H), 3.74 (m, 4H), 3.60 (m, 2H), 3.48 (m, 2H). ¹³C NMR (100 MHz, DMSO-d₆) δ 70.9, 61.39, 61.04, 60.25, 58.54, 57.80.

### Synthesis of 1-amino-3-(4-morpholinyl)-2-propanol (Racemic)

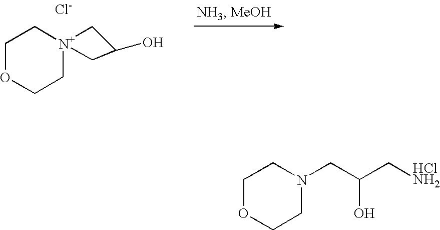

To a 3L 1-neck round bottom flask with a magnetic stir bas was charged 2-hydroxy-7-oxa-4-azoniaspiro[3.5]nonane chloride (150 g, 835 mmole) followed by 23 wt. % anhydrous ammonia in methanol (2120 ml). The flask was stoppered and the resulting clear solution was stirred at 20-23° C. for 18 hours. GC under the conditions above showed no remaining starting material. The stopper was removed and the ammonia allowed to bubble out of the solution for 30 minutes. The flask was then transferred to a rotoevaporated and concentrated to a white solid with 45° C. bath and full house vacuum. ¹H NMR (400 MHz, DMSO-d₆) δ 3.57 (dd, 2H), 3.3-3.5 (m, 6H), 2.59 (m, 2H), 2.2-2.4 (m, 6H); ¹³C NMR (100 MHz DMSO-d₆) δ 70.8, 67.1, 60.1, 53.8, 48.1.

Following the procedure described in Example 3 above but substituting 2-(RS)-1-amino-3-morpholin-4-yl-propan-2-ol with 2-(S)-1-amino-3-morpholin-4-yl-propan-2-ol prepared as described below the desired compound 5-[5-fluoro-2-oxo-1,2-dihydro-indol-(3Z)-ylidenemethyl]-2,4-dimethyl-1H-pyrrole-3-carboxylic acid (2-(S)-hydroxy-3-morpholin-4-yl-propyl)-amide was obtained.

### Synthesis of 1-amino-3-(4-morpholinyl)-2-propanol (Non-Racemic)

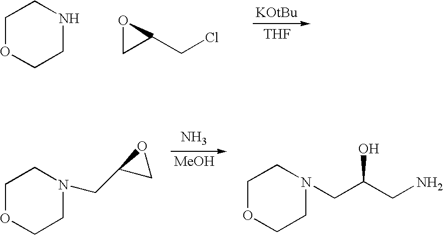

To 1L 3-neck round bottom flask, fitted with mechanical stirring, thermocouple and addition funnel, was charged morpholine (91.5 g, 91.5 ml, 1.05 mole, 1.0 eq.) and 45 ml of t-butanol. The solution was stirred rapidly while adding R-epichlorohydrin (10 g, 84.5 ml, 1.08 mole. 1.03 eq.) from the addition funnel over about 30 minutes. The temperature was monitored and when the pot temperature reached 27° C., the reaction was cooled with an ice water bath. The clear solution was stirred for 18 hours. The reaction was assayed by GC (dilute 5 drops of reaction mixture into 1 ml of ethanol and inject onto a 15 m DB-5 capillary GC column with the following run parameters, Injector 250° C., detector 250° C., initial oven temperature 28° C. warming to 250° C. at 10° C. per minute). The reaction was complete with less than 3% morpholine remaining. The solution was cooled to 10° C. and a 20 wt % solution of potassium t-butoxide in THF (576 g) was added dropwise keeping the temperature less than 15° C. The resulting white slurry was stirred at 10-15° C. for 2 hours and checked by GC using the above conditions. None of the chlorohydrin could be observed. The mixture was concentrated on the rotoevaporated using 50° C. bath and full house vacuum. The resulting mixture was diluted with water (500 ml) and methylene chloride. The phases were separated and the aqueous phase washed with methylene chloride (500 ml). The combined organic layers were dried over sodium sulfate and concentrated to a clear, colorless oil. This provided 145 g, 97% yield of the epoxide. ¹H NMR (400 MHZ, DMSO-d₆) δ 3.3 (dd, 4H), 3.1 (m, 1H), 2.6 (dd, 1H), 2.5 (dd, 1H), 2.4 (m, 4H), 2.2 (dd, 2H); ¹³C NMR (100 MHZ, DMSO-d₆) δ 65.4, 60.1, 53.1, 48.9, 43.4.

The above crude epoxide was charged to a 3L 1-neck round bottom flask with a magnetic stir bar. Anhydrous ammonia in methanol (24% w/w 2.5L) was added, the flask was stoppered and the mixture stirred at room temperature for 24 hours. GC under the conditions above showed no remaining starting material. The stopper was removed and the ammonia allowed to bubble out of the solution for 30 minutes. The flask was then transferred to a rotoevaporated and concentrated to a clear colorless oil with 45° C. bath and full house vacuum. This provided 124 g of product. ¹H NMR (400 MHz, DMSO-d₆) δ 3.57 (dd, 2H), 3.3-3.5 (m, 6H), 2.59 (m, 2H), 2.2-2.4 (m, 6H); ¹³C NMR (100 MHz, DMSO-d₆) δ 70.8, 67.1, 60.1, 53.8, 48.1.

### Synthesis of 1-amino-3-(4-morpholinyl)-2-(S)-propanol

To 1L 3-neck round bottom flask, fitted with mechanical stirring, thermocouple and addition funnel, was charged morpholine (91.5 g, 91.5 ml, 1.05 mole, 1.0 eq.) and 200 ml of methanol. The solution was stirred rapidly while adding R-epichlorohydrin (100 g, 84.5 ml, 1.08 mole, 1.03 eq.) from the addition funnel over about 30 minutes. The temperature was monitored and when the pot temperature reached 27° C., the reaction was cooled with an ice water bath. The clear solution was stirred for 18 hours. The reaction was assayed by GC (dilute 5 drops of reaction mixture into 1 ml of ethanol and inject onto a 15 m DB-5 capillary GC column with the following run parameters, Injector 250° C., detector 250° C., initial oven temperature 28° C. warming to 250° C. at 10° C. per minute.) The reaction was complete with less than 3% morpholine remaining. The solution was cooled to 110° C. and a 25 wt. % solution of sodium methoxide in methanol (233 g, 1.08 mole, 247 ml) was added dropwise keeping the temperature less than 15° C. The resulting white slurry was stirred at 10-15° C. for 2 hours and checked by GC using the above conditions. None of the chlorohydrin could be observed. The mixture was concentrated on the rotoevaporator using 50° C. bath and full house vacuum. The resulting mixture was diluted with water (500 ml) and methylene chloride. The phases were separated and the aqueous phase washed with methylene chloride (500 ml). The combined organic layers were dried over sodium sulfate and concentrated to a clear, colorless oil. This provided 145 g, 97% yield of 1,2-epoxy-3-morpholin-4-ylpropane. ¹H NMR (400 MHz, DMSO-d₆) δ 3.3 (dd, 4H), 3.1 (m, 1H), 2.6 (dd, 1H), 2.5 (dd, 1H), 2.4 (m, 4H), 2.2 (dd, 2H); ¹³C NMR (100 MHz, DMSO-d₆) δ 65.4, 60.1, 53.1, 48.9, 43.4.

The above crude 1,2-epoxy-3-morpholin-4-ylpropane was charged to a 3L 1-neck round bottom flask with a magnetic stir bar. Anhydrous ammonia in methanol (24% w/w 2.5L) was added, the flask was stoppered and the mixture stirred at room temperature for 24 hours. GC under the conditions above showed no remaining starting material. The stopper was removed and the ammonia allowed to bubble out of the solution for 30 minutes. The flask was then transferred to a rotoevaporated and concentrated to a clear colorless oil with 45° C. bath and full house vacuum. This provided 124 g of 1-amino-3-(4-morpholinyl)-2-(S)-propanol.

¹H NMR (400 MHz, DMSO-d₆) δ 3.57 (dd, 2H), 3.3-3.5 (m, 6H), 2.59 (m, 2H), 2.2-2.4 (m, 6H); ¹³C NMR (100 MHz, DMSO-d₆) δ 70.8, 67.1, 60.1, 53.8, 48.1.

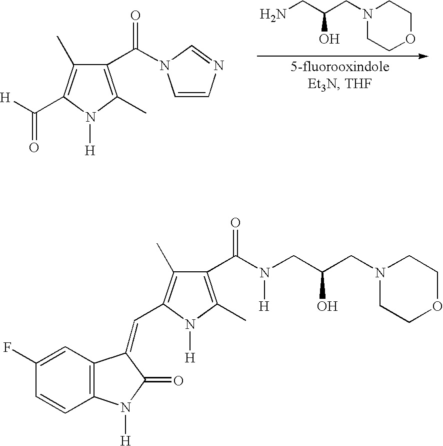

Imidazole amide (7.0 g, 32.3 mmol), amine (15.0 g, 64.6 mmol), 5-fluorooxindole (4.93 g, 32.6 mmol), triethylamine (9.79 g, 96.9 mmol), and THF (88 ml) were mixed and heated to 60° C. A brown solution formed. After stirring for 24 h at 60° C., the yellow slurry was cooled to rt (room temperature) and filtered. The cake was washed with 80 ml THF and dried overnight at 50° C. under house vacuum. A brown solid (23.2 g) was obtained. The solid was slurried in 350 ml water for 5 h at rt and filtered. The cake was washed with 100 ml water and dried at 50° C. under house vacuum overnight. 8.31 g were obtained with 56% chemical yield.

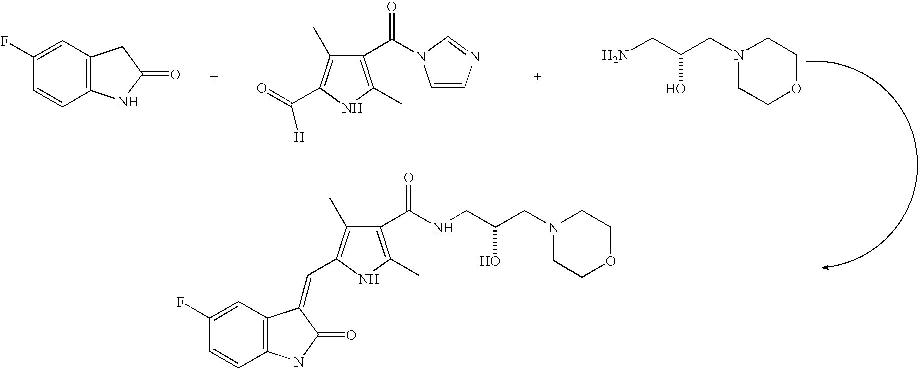

A 0.25L flask fitted with a thermometer, condenser, magnetic stirring, and nitrogen inlet was charged with 4.92 g 5-Fluorooxindole, 7.0 g Imidazole amide, 15.5 g (R)-1-Amino-3-(4-morpholinyl)-2-propanol, 9.78 g Triethylamine and 88 ml Tetrahydrofuran. The mixture was heated to 60° C. for 16.5 hours. The reaction is cooled to ambient temperature and filtered. The solids obtained are slurried (3) three successive times in acetonitrile at 11 ml/g, dried in vacuo for 3.6 g (25.25%). [HPLC, Hypersil BDS, C-18, 5μ, (6:4), Acetonitrile: 0.1M Ammonium Chloride, PHA-571437=4.05 min.] H¹NMR (DMSO): δ 10.86 (1H,bs); 7.75 (1H,d); 7.70 (1H,s); 7.50 (1H,m); 6.88 (2H,m); 4.72 (1H,bs); 3.78 (1H,bs); 3.56 (4H,m); 3.32 (6H,m); 3.15 (1H,m); 2.43 (8H,bm).

### Example 6

### Synthesis of 2,4-dimethyl-5-[2-oxo-1,2-dihydro-indol-(3Z)-ylidenemethyl]-1H-pyrrole-3-carboxylic acid (2-hydroxy-3-morpholin-4-yl-propyl)-amide

5-(2-Oxo-1,2-dihydro-indol-3-ylidenemethyl)-2,4-dimethyl-1H-pyrrole-3-carboxylic acid (113 mg, 0.4 mmol) was condensed with 1-amino-3-morpholin-4-yl-propan-2-ol (74 mg, 0.48 mmol) to precipitate 2,4-dimethyl-5-[2-oxo-1,2-dihydro-indol-(3Z)-ylidenemethyl]-1H-pyrrole-3-carboxylic acid (2-hydroxy-3-morpholin-4-yl-propyl)-amide (77 mg, 45.3%).

¹H NMR (DMSO-d₆) δ 2.27 (m, 1H), 2.32 (m, 1H), 2.40 (m, 4H), 2.40, 2.42 (2×s, 6H, 2×CH₃), 3.15 (s, 1H), 3.32 (m, 1H), 3.55 (m, 4H), 3.77 (m, 1H), 4.74 (d, J=4.8 Hz, 1H, OH), 6.86 (d, J=7.6 Hz, 1H), 6.96 (t, J=7.2 Hz, 1H), 7.10 (t, J=7.6 Hz, 1H), 7.49 (t, J=5.6 Hz, 1H), 7.61 (s, 1H), 7.77 (d, J=8.0 Hz, 1H) (aromatic and vinyl), 10.88 (s, 1H, CONH), 13.62 (s, 1H, NH). LC-MS (m/z) 425.4 (M+1).

### Example 7

### Synthesis of 5-[5-chloro-2-oxo-1,2-dihydro-indol-(3Z)-ylidene-methyl]-2,4-dimethyl-1H-pyrrole-3-carboxylic acid (2-hydroxy-3-morpholin-4-yl-propyl)-amide (Compound 7)

5-(5-Chloro-2-oxo-1,2-dihydro-indol-3-ylidenemethyl)-2,4-dimethyl-1H-pyrrole-3-carboxylic acid (126.6 mg, 0.4 mmol) was condensed with 1-amino-3-morpholin-4-yl-propan-2-ol (74 mg, 0.48 mmol) to precipitate 5-[5-Chloro-2-oxo-1,2-dihydro-indol-(3Z)-ylidenemethyl]-2,4-dimethyl-1H-pyrrole-3-carboxylic acid (2-hydroxy-3-morpholin-4-yl-propyl)-amide (107 mg, 58%).

¹H NMR (DMSO-d₆) δ 2.29 (m, 1H), 2.33 (m, 1H), 2.39(m, 4H), 2.40, 2.42 (2×s, 6H, 2×CH₃), 3.15 (s, 1H), 3.37 (m, 1H), 3.55 (m, 4H), 3.77 (m, 1H), 4.74 (d, J=4.8 Hz, 1H, OH), 6.85 (d, J=8.4 Hz, 1H), 7.11 (dd, J=2.0, 8.0 Hz, 1H), 7.53 (t, J=5.6 Hz, 1H), 7.75 (s, 1H), 7.97 (d, J=2.0 Hz, 1H) (aromatic and vinyl), 10.99 (s, 1H, CONH), 13.62 (s, 1H, NH). LC-MS (m/z) 457.4 (M−1).

### Example 8

### Synthesis of 5-[5-bromo-2-oxo-1,2-dihydro-indol-(3Z)-ylidene-methyl]-2,4-dimethyl-1H-pyrrole-3-carboxylic acid (2-hydroxy-3-morpholin-4-yl-propyl)-amide

5-(5-Bromo-2-oxo-1,2-dihydro-indol-3-ylidenemethyl)-2,4-dimethyl-1H-pyrrole-3-carboxylic acid (72.2 mg, 0.2 mmol) was condensed with 1-amino-3-morpholin-4-yl-propan-2-ol (38 mg, 0.24 mmol) to precipitate 5-[5-Bromo-2-oxo-1,2-dihydro-indol-(3Z)-ylidenemethyl]-2,4-dimethyl-1H-pyrrole-3-carboxylic acid (2-hydroxy-3-morpholin-4-yl-propyl)-amide (55 mg, 55%).

¹H NMR (DMSO-d₆) δ 2.27 (m, 1H), 2.32 (m, 1H), 2.39(m, 4H), 2.41, 2.42 (2×s, 6H, 2×CH₃), 3.13 (s, 1H), 3.35 (m, 1H), 3.55 (m, 4H), 3.77 (m, 1H), 4.74 (d, J=4.4 Hz, 1H, OH), 6.80 (d, J=8.4 Hz, 1H), 7.24 (dd, J=2.0, 8.0 Hz, 1H), 7.51 (t, J=5.6 Hz, 1H), 7.76 (s, 1H), 8.09 (d, J=2.0 Hz, 1H) (aromatic and vinyl), 10.99 (s, 1H, CONH), 13.62 (s, 1H, NH). LC-MS (m/z) 503.4 (M−1).

### Example 9

### Synthesis of 2,4-dimethyl-5-[2-oxo-1,2-dihydro-indol-(3Z)-ylidene-methyl]-1H-pyrrole-3-carboxylic acid (2-hydroxy-3-[1,2,3]triazol-1-yl-propyl)-amide

Step 1

A mixture of 3-[1,2,3]triazole (2.0 g, 29 mmol), epichlorohydrin (3.4 ml, 43.5 mmol) and N,N-diisopropyl-ethylamine (2.6 mL, 15 mmol) in ethanol (50 mL) was stirred at room temperature overnight. After removing the solvents, the residue was purified by flash chromatography (CH₂Cl₂/CH₃OH=100/1-100/2-100/4) to give 1-chloro-3-(1,2,3)-triazol-2-ylpropan-2-ol (2.1 g, 45%). ¹H NMR (CDCl₃) δ 3.52 (m, 2H, OH and CH₂), 3.60 (dd, J=5.2, 11.2 Hz, 1H), 4.36 (m, 1H, CH), 4.68 (m, 2H), 7.67 (s, 2H). MS (m/z) 162.1 (M+1) and 1-chloro-3-(1,2,3)triazol-1-ylpropan-2-ol (2.3 g, 49%). ¹H NMR (CDCl₃) δ 3.56 (s, 1H), 3.57 (s, 1H), 4.35 (m, 1H), 4.53 (dd, J=7.2, 14 Hz, 1H), 4.67 (dd, J=3.8, 14 Hz, 1H), 7.67 (s, 1H), 7.71 (s, 1H). MS (m/z) 162.1 (M+1).

Step 2

1-Chloro-3(1,2,3)triazol-1-ylpropan-2-ol (2.3 g, 13 mmol) was treated with the solution of NH₃ in methanol (25% by weight, 20 mL) at 60° C. overnight in a sealed pressure vessel. After cooling to room temperature, nitrogen was bulbbed into the reaction mixture to remove the ammonia. Evaporation of solvent gave the hydrogen chloride salt of 1-amino-3-(1,2,3)triazol-1-ylpropan-2-ol (2.57 g, 100%).

¹H NMR (DMSO-d₆) δ 2.68 (dd, J=8.8, 12.8 Hz, 1H), 2.97 (dd, J=3.6, 12.8 Hz, 1H), 4.15 (m, 1H), 4.44 (dd, J=6.4, 14 Hz, 1H), 4.57 (dd, J=4.6, 14 Hz, 1H), 5.95 (d, J=5.2 Hz, 1H, OH), 7.77 (s, 1H), 8.01 (brs., 3H, NH₃+), 8.12 (s, 1H). MS (m/z) 143.1 (M+1).

Step 3

5-(2-Oxo-1,2-dihydro-indol-3-ylidenemethyl)-2,4-dimethyl-1H-pyrrole-3-carboxylic acid (113 mg, 0.4 mmol) was condensed with 1-amino-3(1,2,3)triazole-1-yl-propan-2-ol (85 mg, 0.48 mmol) to precipitate 2,4-dimethyl-5-[2-oxo-1,2-dihydro-indol-(3Z)-ylidenemethyl]-1H-pyrrole-3-carboxylic acid (2-hydroxy-3-[1,2,3]triazol-1-yl-propyl)-amide (70 mg, 41%).

¹H NMR (DMSO-d₆) δ 2.45, 2.48 (2×s, 6H, 2×CH₃), 3.35 (m, 2H), 4.02 (m, 1H), 4.32 (dd, J=7.6, 14 Hz, 1H), 4.53 (dd, J=3.4, 14 Hz, 1H), 5.43 (d, J=5.6 Hz, 1H, OH), 6.91 (d, J=7.6 Hz, 1H), 7.01 (t, J=7.6 Hz, 1H), 7.15 (t, J=8.0 Hz, 1H), 7.66 (s, 1H), 7.12 (t, J=5.6 Hz, 1H), 7.74 (s, 1H), 7.77 (d, J=7.6 Hz, 1H), 8.11 (s, 1H), 10.93 (s, 1H, CONH), 13.68 (s, 1H, NH). LC-MS (m/z) 405.4 (M−1).

### Example 10

### Synthesis of 5-[5-fluoro-2-oxo-1,2-dihydro-indol-(3Z)-ylidene-methyl]-2,4-dimethyl-1H-pyrrole-3-carboxylic acid (2-hydroxy-3-[1,2,3]triazol-1-yl-propyl)-amide

5-(5-Fluoro-2-oxo-1,2-dihydro-indol-3-ylidenemethyl)-2,4-dimethyl-1H-pyrrole-3-carboxylic acid (120 mg, 0.4 mmol) was condensed with 1-amino-3(1,2,3)triazol-1-yl-propan-2-ol (85 mg, 0.48 mmol) to precipitate 5-[5-fluoro-2-oxo-1,2-dihydro-indol-(3Z)-ylidenemethyl]-2,4-dimethyl-1H-pyrrole-3-carboxylic acid (2-hydroxy-3-[1,2,3]triazol-1-yl-propyl)-amide (100 mg, 62%).

¹H NMR (DMSO-d₆) δ 2.42, 2.44 (2×s, 6H, 2×CH₃), 3.27 (m, 2H), 3.98 (m, 1H), 4.27 (dd, J=7.6, 14 Hz, 1H), 4.50 (dd, J=3.4, 13.6 Hz, 1H), 5.38 (d, J=5.6 Hz, 1H, OH), 6.82 (dd, J=4.4, 8.4 Hz, 1H), 6.91 (td, ²J=2.4, ³J=9.0 Hz, 1H), 7.70 (m, 3H), 7.75 (dd, J=2.4, 9.2 Hz, 1H), 8.11 (s. 1H), 10.93 (s, 1H, CONH), 13.73 (s, 1H, NH). LC-MS (m/z) 423.4 (M−1).

### Example 11

### Synthesis of 5-[5-chloro-2-oxo-1,2-dihydro-indol-(3Z)-ylidene-methyl]-2,4-dimethyl-1H-pyrrole-3-carboxylic acid (2-hydroxy-3-[1,2,3]triazol-1-yl-propyl)-amide

5-(5-Chloro-2-oxo-1,2-dihydro-indol-3-ylidenemethyl)-2,4-dimethyl-1H-pyrrole-3-carboxylic acid (126.6 mg, 0.4 mmol) was condensed with 1-amino-3(1,2,3)triazole-1-yl-propan-2-ol (85 mg, 0.48 mmol) to precipitate 5-[5-Chloro-2-oxo-1,2-dihydro-indol-(3Z)-ylidenemethyl]-2,4-dimethyl-1H-pyrrole-3-carboxylic acid (2-hydroxy-3-[1,2,3]triazol-1-yl-propyl)-amide (48 mg, 27%).

¹H NMR (DMSO-d₆) δ 2.42, 2.44 (2×s, 6H, 2×CH3), 3.27 (m, 2H), 3.99 (m, 1H), 4.28 (dd, J=7.8, 14 Hz, 1H), 4.51 (dd, J=3.2, 14 Hz, 1H), 5.39 (d, J=6.0 Hz, 1H, OH), 6.85 (d, J=8.4 Hz, 1H), 7.12 (dd, J=2.0, 8.2 Hz, 1H), 7.70 (m, 2H), 7.74 (s, 1H), 7.97 (d, J=2.0 Hz, 1H), 8.07 (s, 1H), 10.99 (s, 1H, CONH), 13.65 (s, 1H, NH). LC-MS (m/z) 439.4 (M−1).

### Example 12

### Synthesis of 5-[5-bromo-2-oxo-1,2-dihydro-indol-(3Z)-ylidene-methyl]-2,4-dimethyl-1H-pyrrole-3-carboxylic acid (2-hydroxy-3-[1,2,3]triazol-1-yl-propyl)-amide

5-(5-Bromo-2-oxo-1,2-dihydro-indol-3-ylidenemethyl)-2,4-dimethyl-1H-pyrrole-3-carboxylic acid (144.4 mg, 0.4 mmol) was condensed with 1-amino-3(1,2,3)triazole-1-yl-propan-2-ol (85 mg, 0.48 mmol) to precipitate 5-[5-bromo-2-oxo-1,2-dihydro-indol-(3Z)-ylidenemethyl]-2,4-dimethyl-1H-pyrrole-3-carboxylic acid (2-hydroxy-3-[1,2,3]triazol-1-yl-propyl)-amide 130 mg, 67%).

¹H NMR (DMSO-d₆) δ 2.41, 2.44 (2×s, 6H, 2×CH₃), 3.27 (m, 2H), 3.99 (m, 1H), 4.28 (dd, J=7.6, 14 Hz, 1H), 4.50 (dd, J=3.6, 14 Hz, 1H), 5.40 (d, J=5.6 Hz, 1H, OH), 6.81 (d, J=8.4 Hz, 1H), 7.24 (dd, J=2.0, 8.0 Hz, 1H), 7.70 (m, 2H), 7.77 (s, 1H), 8.07 (s. 1H), 8.10 (d, J=1.6 Hz, 1H), 11.0 (s, 1H, CONH), 13.64 (s, 1H, NH). LC-MS (m/z) 485.4 (M−1).

### Example 13

### Synthesis of 5-(5-Fluoro-2-oxo-1,2-dihydroindol-3-ylidenemethyl)-2,4-dimethyl-1H-pyrrole-3-carboxylic acid (2-diethylamino-ethyl)amide (Compound 1)

5-Fluoro-1,3-dihydroindol-2-one (0.54 g, 3.8 mmol) was condensed with 5-formyl-2,4-dimethyl-1H-pyrrole-3-carboxylic acid (2-diethylaminoethyl)amide to give 0.83 g (55%) of the title compound as a yellow green solid.

Alternative Synthesis of 5-(5-Fluoro-2-oxo-1,2-dihydroindol-3-ylidenemethyl)-2,4-dimethyl-1H-pyrrole-3-carboxylic acid (2-diethylamino-ethyl)amide

Hydrazine hydrate (55%, 3000 mL) and 5-fluoro-isatin (300 g) were heated to 100° C. An additional 5-fluoro-isatin (500 g) was added in portions (100 g) over 120 minutes with stirring. The mixture was heated to 110° C. and stirred for 4 hours. The mixture was cooled to room temperature and the solids collected by vacuum filtration to give crude (2-amino-5-fluoro-phenyl)-acetic acid hydrazide (748 g). The hydrazide was suspended in water (700 mL) and the pH of the mixture adjusted to <pH 3 with 12 N hydrochloric acid. The mixture was stirred for 12 hours at room temperature. The solids were collected by vacuum filtration and washed twice with water. The product was dried under vacuum to give 5-fluoro-1,3-dihydro-indol-2-one (600 g, 73% yield) as a brown powder. ¹H-NMR (dimethylsulfoxide-d_{g}) δ 3.46 (s, 2H, CH₂), 6.75, 6.95, 7.05 (3×m, 3H, aromatic), 10.35 (s, 1H, NH). MS m/z 152 [M+1].

3,5-Dimethyl-1H-pyrrole-2,4-dicarboxylic acid 2-tert-butyl ester 4-ethyl ester (2600 g) and ethanol (7800 mL) were stirred vigorously while 10 N hydrochloric acid (3650 mL) was slowly added. The temperature increased from 25° C. to 35° C. and gas evolution began. The mixture was warmed to 54° C. and stirred with further heating for one hour at which time the temperature was 67° C. The mixture was cooled to 5° C. and 32 L of ice and water were slowly added with stirring. The solid was collected by vacuum filtration and washed three times with water. The solid was air dried to constant weight to give of 2,4-dimethyl-1H-pyrrole-3-carboxylic acid ethyl ester (1418 g, 87% yield) as a pinkish solid. ¹H-NMR (dimethylsulfoxide-d₆) δ 2.10, 2.35 (2×s, 2×3H, 2×CH₃), 4.13 (q, 2H, CH₂), 6.37 (s, 1H, CH), 10.85 (s, 1H, NH). MS m/z 167 [M+1].

Dimethylformamide (322 g) and dichloromethane (3700 mL) were cooled in an ice bath to 4° C. and phosphorus oxychloride (684 g) was added with stirring. Solid 2,4-dimethyl-1H-pyrrole-3-carboxylic acid ethyl ester (670 g) was slowly added in aliquots over 15 minutes. The maximum temperature reached was 18° C. The mixture was heated to reflux for one hour, cooled to 10° C. in an ice bath and 1.6 L of ice water was rapidly added with vigorous stirring. The temperature increased to 15° C. 10 N Hydrochloric acid (1.6 L) was added with vigorous stirring. The temperature increased to 22° C. The mixture was allowed to stand for 30 minutes and the layers allowed to separate. The temperature reached a maximum of 40° C. The aqueous layer was adjusted to pH 12-13 with 10 N potassium hydroxide (3.8 L) at a rate that allowed the temperature to reach and remain at 55° C. during the addition. After the addition was complete the mixture was cooled to 10° C. and stirred for 1 hour. The solid was collected by vacuum filtration and washed four times with water to give 5-formyl-2,4-dimethyl-1H-pyrrole-3-carboxylic acid ethyl ester (778 g, 100% yield) as a yellow solid. ¹H-NMR (DMSO-d) δ 1.25 (t, 3H, CH₃), 2.44, 2.48 (2×s, 2×3H, 2×CH₃), 4.16 (q, 2H, CH₂), 9.59 (s, 1H, CHO), 12.15 (br s, 1H, NH). MS m/z 195 [M+1].

5-Formyl-2,4-dimethyl-1H-pyrrole-3-carboxylic acid ethyl ester (806 g), potassium hydroxide (548 g), water (2400 mL) and methanol (300 mL) were refluxed for two hours with stirring and then cooled to 8° C. The mixture was extracted twice with dichloromethane. The aqueous layer was adjusted to pH 4 with 1000 mL of 10 N hydrochloric acid keeping the temperature under 15° C. Water was added to facilitate stirring. The solid was collected by vacuum filtration, washed three times with water and dried under vacuum at 50° C. to give 5-formyl-2,4-dimethyl-1H-pyrrole-3-carboxylic (645 g, 93.5% yield) acid as a yellow solid. ¹H-NMR (DMSO-d₆) δ 2.40, 2.43 (2×s, 2×3H, 2×CH₃), 9.57 (s, 1H, CHO), 12.07 (br s, 2H, NH+COOH). MS m/z 168 [M+1].

5-Formyl-2,4-dimethyl-1H-pyrrole-3-carboxylic acid (1204 g) and 6020 mL of dimethylformamide were stirred at room temperature while 1-(3-dimethyl-aminopropyl-3-ethylcarbodiimide hydrochloride (2071 g), hydroxybenzotriazole (1460 g), triethylamine (2016 mL) and diethylethylenediamine (1215 mL) were added. The mixture was stirred for 20 hours at room temperature. The mixture was diluted with 3000 mL of water, 2000 mL of brine and 3000 mL of saturated sodium bicarbonate solution and the pH adjusted to greater than 10 with 10 N sodium hydroxide. The mixture was extracted twice with 5000 mL each time of 10% methanol in dichloromethane and the extracts combined, dried over anhydrous magnesium sulfate and rotary evaporated to dryness. The mixture was with diluted with 1950 mL of toluene and rotary evaporated again to dryness. The residue was triturated with 3:1 hexane:diethyl ether (4000 mL). The solids were collected by vacuum filtration, washed twice with 400 mL of ethyl acetate and dried under vacuum at 34° C. for 21 hours to give 5-formyl-2,4-dimethyl-1H-pyrrole-3-carboxylic acid (2-diethylamino-ethyl)-amide (819 g, 43% yield) as a light brown solid. ¹H-NMR (dimethylsulfoxide-d₆) δ 0.96 (t, 6H, 2×CH₃), 2.31, 2.38 (2×s, 2×CH₃), 2.51 (m, 6H 3×CH₂), 3.28 (m, 2H, CH₂), 7.34 (m, 1H, amide NH), 9.56 (s, 1H, CHO), 11.86 (s, 1H, pyrrole NH). MS m/z 266 [M+1].

5-Formyl-2,4-dimethyl-1H-pyrrole-3-carboxylic acid (2-diethylaminoethyl)-amide (809 g), 5-fluoro-1,3-dihydro-indol-2-one (438 g), ethanol (8000 mL) and pyrrolidine (13 mL) were heated at 78° C. for 3 hours. The mixture was cooled to room temperature and the solids collected by vacuum filtration and washed with ethanol. The solids were stirred with ethanol (5900 mL) at 72° C. for 30 minutes. The mixture was cooled to room temperature. The solids were collected by vacuum filtration, washed with ethanol and dried under vacuum at 54° C. for 130 hours to give 5-[5-fluoro-2-oxo-1,2-dihydro-indol-(3Z)-ylidenemethyl]-2,4-dimethyl-1H-pyrrole-3-carboxylic acid (2-diethylamino-ethyl)-amide (1013 g, 88% yield) as an orange solid. ¹H-NMR (dimethylsulfoxide-d₆) δ 0.98 (t, 6H, 2×CH3), 2.43, 2.44 (2×s, 6H, 2×CH3), 2.50 (m, 6H, 3×CH2), 3.28 (q, 2H, CH₂), 6.84, 6.92, 7.42, 7.71, 7.50 (5×m, 5H, aromatic, vinyl, CONH), 10.88 (s, 1H, CONH), 13.68 (s, 1H, pyrrole NH). MS m/z 397 [M-1].

The malic salt of 5-(5-Fluoro-2-oxo-1,2-dihydroindol-3-ylidenemethyl)-2,4-dimethyl-1H-pyrrole-3-carboxylic acid (2-diethylamino-ethyl)amide can be prepared according to the disclosure of U.S. patent application Ser. No. 10/281,985, filed Aug. 13, 2002, which claims priority to U.S. Patent Provisional Application No. 60/312,353, filed Aug. 15, 2001, which is incorporated by reference in its entirety.

Synthesis of 5-(5-bromo-2-oxo-1,2-dihydro-indol-3-ylidenemethyl)-2,4-dimethyl-1H-pyrrole-3-carboxylic acid, 5-(5-chloro-2-oxo-1,2-dihydro-indol-3-ylidenemethyl)-2,4-dimethyl-1H-pyrrole-3-carboxylic acid, 5-(2-oxo-1,2-dihydro-indol-3-ylidenemethyl)-2,4-dimethyl-1H-pyrrole-3-carboxylic acid is described in Ser. No. 09/783,264 filed on Feb. 14, 2001, titled “PYRROLE SUBSTITUTED 2-INDOLINONE—PROTEIN KINASE INHIBITORS”, the disclosure of which is incorporated herein in its entirety.

### Example 14

### Synthesis of 5-(5-Fluoro-2-oxo-1,2-dihydro-indol-3-ylidenemethyl)-2,4-dimethyl-1H-pyrrole-3-carboxylic acid (2-pyrrolidin-1-yl-ethyl)-amide (Compound 2)

5-Fluoro-1,3-dihydro-indolin-2-one was condensed with 5-formyl-2,4-dimethyl-1H-pyrrole-3-carboxylic acid (2-pyrrolidin-1-yl-ethyl)-amide to yield the title compound.

MS+ve APCI 397 [M+1].

### Exmaple 15

### Synthesis of 5-(5-Fluoro-2-oxo-1,2-dihydro-indol-(3Z)-ylidenemethyl)-2,4-dimethyl-1H-pyrrole-3-carboxylic acid (2-ethylamino-ethyl)-amide (Compound 8)

5-Formyl-2,4-dimethyl-1H-pyrrole-3-carboxylic acid (2-ethylamino-ethyl)-amide (99 g), ethanol (400 ml), 5-fluoro-2-oxindole (32 g) and pyrrolidine (1.5 g) were refluxed for 3 hours with stirring. The mixture was cooled to room temperature and the solids collected by vacuum filtration. The solids were stirred in ethanol at 60° C., cooled to room temperature and collected by vacuum filtration. The product was dried under vacuum to give 5-(5-Fluoro-2-oxo-1,2-dihydro-indol-(3Z)-ylidenemethyl)-2,4-dimethyl-1H-pyrrole-3-carboxylic acid (2-ethylamino-ethyl)-amide (75 g, 95% yield). ¹H-NMR (dimethylsulfoxide-d₆) δ 1.03 (t, 3H, CH₃), 2.42, 2.44 (2×s, 6H, 2×CH₃), 2.56 (q, 2H, CH₂), 2.70, 3.30 (2×t, 4H, 2×CH₂), 6.85, 6.92, 7.58, 7.72, 7.76 (5×m, 5H, aromatic, vinyl, and CONH), 10.90 (br s, 1H, CONH), 13.65 (br s, 1H, pyrrole NH).

MS m/z 369 [M+1].

### Example 16

### Synthesis of 5-(5-Fluoro-2-oxo-1,2-dihydro-indol-3-ylidenemethyl)-2,4-dimethyl-1H-pyrrole-3-carboxylic acid (2-morpholin-4-yl-ethyl)-amide (Compound 3)

5-Fluoro-1,3-dihydro-indolin-2-one was condensed with 5-formyl-2,4-dimethyl-1H-pyrrole-3-carboxylic acid (2-morpholin-1-yl-ethyl)-amide to yield the title compound.

### BIOLOGICAL EXAMPLES

### Example 17

### 5-(5-Fluoro-2-oxo-1,2-dihydro-indol-3-ylidenemethyl)-2,4-dimethyl-1H-pyrrole-3-carboxylic acid (2-diethylamino-ethyl)-amide (compound 1) inhibits phosphorylation of CSF1R

3T3-huCSF1R cells were starved overnight (RPMI 1640/0.1% FBS), and then resuspended in fresh RPMI 1640 containing 0.1% FBS±compound 1, using 20 million cells per condition in 6-well plates. Cells were treated at 37° C. for 2 hours with compound 1, then stimulated for 10 mins with human M-CSF at 100 ng/ml (R&D Systems, Minneapolis, Minn.). Cells were lysed immediately after stimulation and lysates spun at 4° C. for 20 mins. Supernatant was transferred to new microfuge tubes. For each sample, 500 μg of total protein was immunoprecipitated overnight with a rabbit polyclonal antibody to bead-conjugated human CSF1R (Santa Cruz Biotechnology, CA). A p-CSF1R Western blot was then performed, using anti-phospho-CSF1R (Tyr₇₂₃) antibody (Cell Signaling Technology, Beverly, Mass.) at 1:1000 dilution. Antibody to actin was used as a control for total protein, as the antibody to CSF1R does not recognize p-CSF1R well.

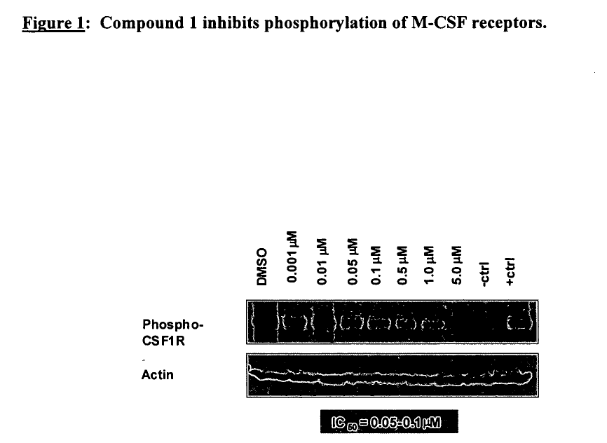

The results, shown in FIG. 1, demonstrate that compound 1 inhibits CSF1R phosphorylation in a dose-dependent manner. As the positive control lane shows, absent inhibition, M-CSF stimulates CSF1R phosphorylation. However, with increasing doses of compound 1, CSF1R phosphorylation declined. The IC50 for inhibition of CSF1R when expressed by NIH3T3 cells by compound 1 was 50-100 nM.

### Example 18

### 5-(5-Fluoro-2-oxo-1,2-dihydro-indol-3-ylidenemethyl)-2,4-dimethyl-1H-pyrrole-3-carboxylic acid (2-diethylamino-ethyl)-amide (compound 1) Inhibits Murine Osteoclast Development in Vitro

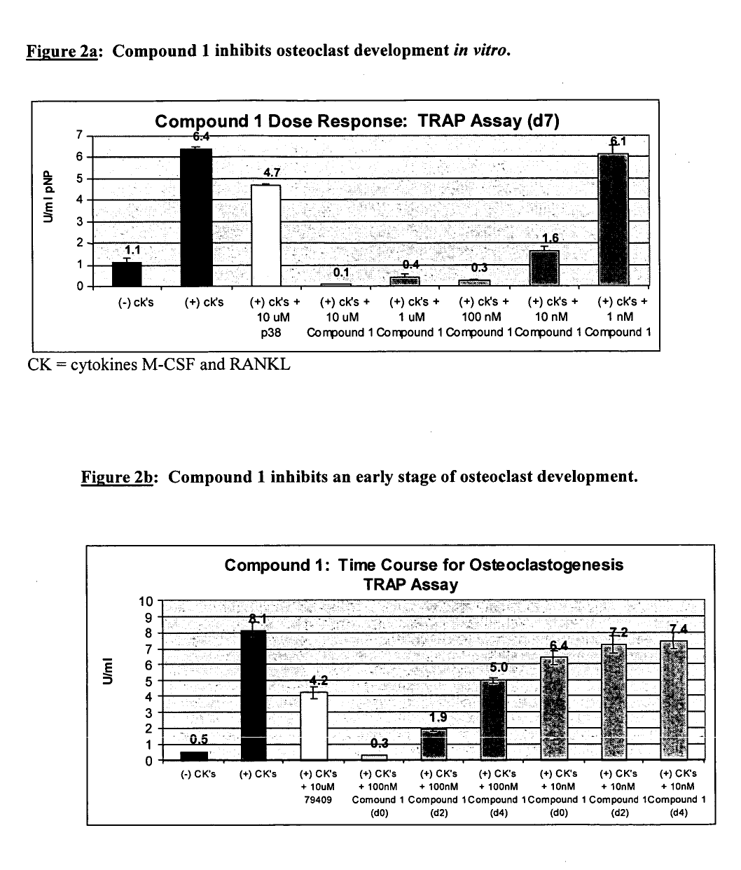

Bone marrow cells were isolated from female Balb/c mice and cultured either in medium alone or with addition of 10 ng/ml murine M-CSF and 100 ng/ml murine RANK ligand (RANKL) from day 0 to induce osteoclast development. To cultures with cytokines, different concentrations of compound 1 were added, from 1 nM to 10 μM. Osteoclast development on day 7 was assessed by colorimetric quantitation of tartrate resistant acid phosphatase activity (TRAP) as well as counting TRAP positive cells with >3 nuclei. As FIG. 2a shows, compound 1 inhibits the development of osteoclasts at 10-100 nM concentrations.

A similar study was performed, adding cytokines at day 0 but varying the time of addition of compound 1 (D0, D2 or D4). TRAP staining and quantitation were performed at day 6. This study demonstrated that the mechanism of action was inhibition of M-CSF effect (early phase development), and not RANKL effect (late phase development. FIG. 2b summarizes the data, and shows that at concentrations of both 100 nM and 10 nM, the later precursor cells are exposed to compound 1, the less inhibitory effect the compound exhibits.

### Example 19

### 5-(5-Fluoro-2-oxo-1,2-dihydro-indol-3-ylidenemethyl)-2,4-dimethyl-1H-pyrrole-3-carboxylic acid (2-diethylamino-ethyl)-amide (compound 1) Inhibits Breast Cancer Growth in Vivo

The MDA-MB-435-HAL-luc breast cancer xenograft model was used to confirm significant inhibition of the growth of breast cancer bone metastases. The presence of live tumor cells was monitored by bioluminescence imaging with the Xenogen IVIS™ system. Mice treated with compound 1 showed a significant reduction in live tumor.

Cell Lines

The human breast carcinoma cell line 435/HAL was obtained (Pharmacia Corp., St. Louis, Mo.). This line was isolated using an in vivo selection procedure to identify a derivative of MDA-MB-435 human breast carcinoma cell line that exhibited increased primary tumor growth rate and increased pulmonary metastasis in vivo (30). Stable transfection of 435/HAL cells with luciferase (light emitting enzyme of firefly Photinus pyralis) was then performed. The cells were cotransfected with pGL3-control (Promega, Madison, Wis.) and pTK-Hygro (Clontech, Palo Alto, Calif.) at a 1:4 ratio using Lipofectamine 2000 (Invitrogen, Carlsbad, Calif.). Cells were maintained in Hygromycin (200 μg/ml) (Invitrogen) and resistant colonies isolated by ring cloning. Hygro-resistant colonies were screened for luciferase expression using Promega brite-glo reagent, normalized as RLU/μg protein. A subclone with the highest luciferase activity was selected, which we refer to as ‘435/HAL-luc.’

These cells were cultured in RPMI 1640 supplemented with 10% fetal bovine serum, 2 mM glutamine, 1 mM sodium pyruvate (Life Technologies Inc., Gaithersburg, Md.), and maintained routinely in a humidified chamber at 37° C. and 5% carbon dioxide. Cells were harvested from culture flasks during exponential growth, washed once with sterile phosphate-buffered saline (PBS), counted, and resuspended in PBS to a suitable concentration prior to implantation.

Mice

Female athymic nu/nu mice were obtained (Charles River Laboratories, Wilmington, Mass.). The Mice were housed under pathogen-free conditions in microisolator cages with sterile rodent chow and water available ad libitum. All xenograft animal studies were performed in an AAALAC, International accredited vivarium and in accordance with the Institute of Laboratory Animal Research Guide for the Care and Use of Laboratory Animals (National Institutes of Health, Bethesda, Md.). Mice were approximately 8 weeks old when cells were implanted via the left ventricle of the heart to evaluate growth in bone, and 10-11 weeks old when tumor pieces were implanted into the #2 mammary fat pad to evaluate orthotopic growth.

Breast Cancer Detection in Mice using the IVIS™ Imaging System

Mice were injected intraperitoneally with 150 mg/kg of luciferin (Xenogen Corp., Alameda, Calif.), followed by anesthetization with Ketamine/Xylazine five minutes later. After another five minutes, mice were imaged using an intensified charge-coupled device (ICCD) camera in the Xenogen IVIS™ imaging system (Xenogen Corp.) to evaluate the bioluminescence of cancer in the mouse. Briefly, mice were placed on the temperature-controlled bed of the imaging chamber and a gray-scale whole body image of the ventral side of the mice was captured, followed by an overlay of a bioluminescence map representing the spatial distribution of photons detected from cleaved luciferin in the cancer cells expressing luciferase. A final image at day 46 was taken of the dorsal side of the mice to monitor growth of tumor in the spine. The bioluminescent signal was quantified by counting the pixels within the area drawn around each site of photon emission, using a customized version of the IGOR Pro version 4.0 Software (WaveMetrics, Inc., Lake Oswego, Oreg.) called Living Image version 2.11 (Xenogen Corporation, Alameda, Calif.). The sum of all detected photon counts within a region of interest containing the entire cancer lesion was determined. For evaluation of tumor growth inhibition, the Student's t test was used to assess differences in photon emission readings between treated and control groups (p<0.05 was considered significant).

### Example 20

### 5-(5-Fluoro-2-oxo-1,2-dihydro-indol-3-ylidenemethyl)-2,4-dimethyl-1H-pyrrole-3-carboxylic acid (2-diethylamino-ethyl)-amide (compound 1) Inhibits Osteolysis Induced by Breast Cancer Metastasis in Vivo

Compound 1 Treatment in an Experimental Bone Metastasis Model

Athymic mice were inoculated with 3×10⁶ 435/HAL-luc cells into the left ventricle of the heart on day 0. Twenty days later, mice were imaged using the IVIS™ imaging system and placed into two matched groups of 16 mice based on photon emission, a measure of bioluminescence of the cancer. The next day, mice bearing established 435/HAL-luc tumor in bone were administered 80 or 40 mg per kg of compound 1 or CMC vehicle once daily by gavage to the end of the study (21 days). Mice were imaged approximately once a week. By 41 days after implantation, mice from the control group became cachectic and exhibited signs of hind limb paralysis triggering the end of the study. The femur, mandible and spines were collected from mice treated with either compound 1 or its vehicle, and fixed in Streck's Tissue Fixative prior to bone density scanning and histological analysis. Serum was also collected for measurement of collagen breakdown product pyridinoline (PYD) in the circulation.

Compound 1 Inhibited Growth of 435/HAL-luc Osseous Metastasis

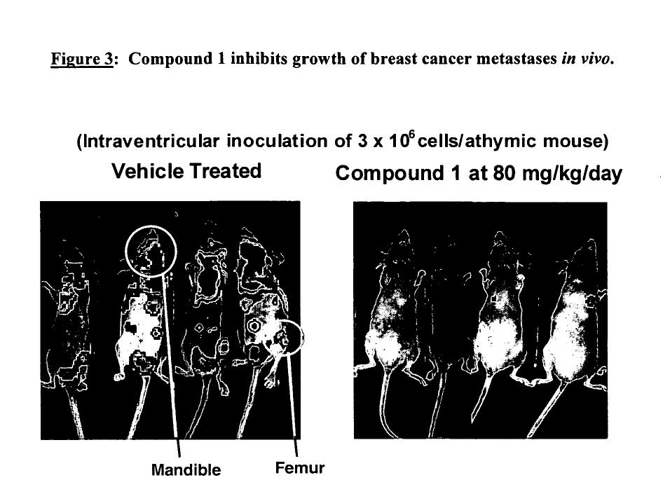

This experiment confirmed that compound 1 could significantly inhibit growth of breast cancer metastases in bone. During therapy, ventral bioluminescence whole body images were acquired to analyze tumor growth in long bones and mandibles. Compound 1 greatly decreased the photon count emission from the mouse bones at both doses (FIG. 3). Photon emission from the thoracic region is likely to be due to tumor cell deposition into the pericardial or pleural cavities at the time of intracardiac injection. Prior to termination, both ventral and dorsal images were taken to also analyze spinal metastases. Regions of interest (ROI) that captured tumor growth in different sites were analyzed separately. Combined data for the change in photon emission from long bones (femur and tibia) and mandibles over time is shown in FIG. 3. Tumor growth in bone was significantly inhibited by compound 1 (day 41: 89% inhibition, p=0.001).

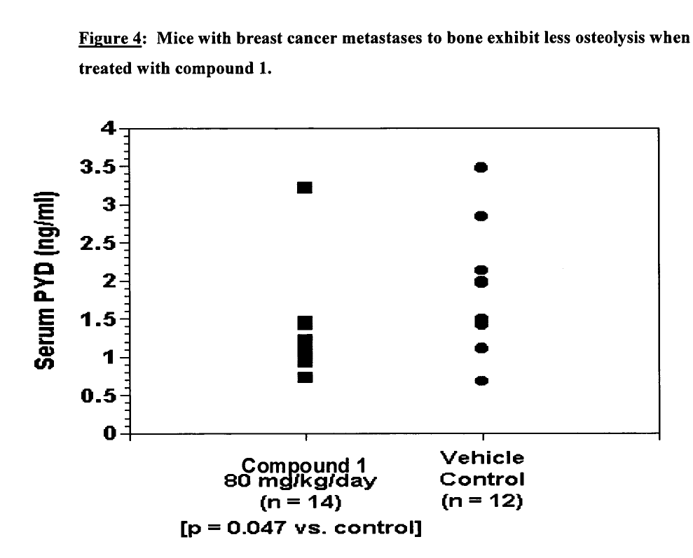

Serum PYD ELISA: The parental breast carcinoma line MDA-MB-435, is very well characterized as having osteolytic activity (32). Measurement of serum levels of the collagen breakdown product pyridinoline (PYD) is an established assay for osteolytic activity that correlates significantly with the volume of bone metastasis in a rat model (29, 31, 33). Serum samples were collected, aliquoted and frozen at −80° C. until analysis. Serum PYD was measured using a competitive enzyme immunoassay kit following the manufacturer's protocol (Serum PYD, Quidel 8019, San Diego, Calif.). Samples were measured in duplicate. Results from 12 vehicle treated and 14 compound 1 treated mice showed that serum PYD levels were significantly reduced by 30% in mice receiving compound 1 treatment as compared to vehicle treated mice (p=0.047). The mean value was 1.8+/−0.21 ng/ml in vehicle treated versus 1.3+/−0.16 ng/ml in mice treated with 80 mg per kg compound 1. FIG. 4 shows the distribution of data points for this experiment.

It will be apparent to those skilled in the art that various modifications and variations can be made in the methods and compositions of the present invention without departing from the spirit or scope of the invention. It is intended that the present invention covers such modifications and variations, provided they come within the scope of the appended claims and their equivalents.

### References

The disclosure of each of the following references, cited above, is incorporated herein by reference.

1. Mundy, Clin. Orthop. 324: 24-28 (1996).

2. Mundy, J. Bone Miner. Res., 8: S505-10 (1993).

3. Tanaka et al., J. Clin. Invest., 91: 257-263 (1993).

4. Yasuda, H., Proc. Natl. Acad. Sci. USA., 95: 3597-3602 (1998).

5. Friedman et al., Science, 150: 1465-67 (1965).

6. Takahashi et al., J Clin Invest, 95:167-171 (1995).

7. Fisher et al., Endocrinology, 132:1411-1413 (1993).

8. Davies et al., J Cell Biol, 109:1817-1826 (1989).

9. Hentunen et al., J Bone Miner Res, 6:1091-1097 (1991).

10. Weber et al., J. Bone Miner Res, 5:401-410 (1990).

11. Coleman et al, Cancer Treatment Rev, 19:79-103 (1993).

12. Hall et al., Calcif. Tiss. Intl., 57:463-465 (1995).

13. Brandi et al., Proc Natl Acad Sci USA, 92:2954-2958 (1995).

14. Yoneda et al., Cancer Res, 55:1989-1993 (1995).

15. Jilka et al. Science, 257:88-91, (1992).

16. O'Neill et al., Bone, 13:23-27 (1992).

17. Quinn et al., J. Pathology, 184: 31-36 (1998).

18. Clohisy et al., Clinical Orthopaedics and Related Res., 373: 104-14 (2000).

19. Mancino et al., J. of Surgical Res., 100: 18-24 (2001).

20. Pederson et al., Cancer Res., 59: 5849 (1999)

21. Hunt et al., British J. Cancer, 85(1): 78-84 (2001).

22. Yee et al., Anticancer Res., 20(6B): 4379 (2000).

23. Kacinski et al., Oncogene, 6: 941 (1991).

24. Flick et al., Oncogene, 14: 2553 (1997).

25. Maher et al., Clin. Cancer Res., 4: 1851 (1998).

26. Kacinski, Ann. Med., 27: 79 (1995).

27. Kacinski, Mol. Reprod. Dev., 46: 71 (1997).

28. Toy et al., Gynecologic Oncology, 80: 194-200 (2001)

29. Demers et al. Cancer Supplement 88:2919 (2000).

30. Schmidt et al., Clin. & Exper. Metastasis, 17: 537-44 (1999).

31. Robins et al., Clin. Chem., 42: 1621-26 (1996).

32. Hunt et al., Br. J. Cancer 85: 78-84 (2001).

33. Tamura et al., Breast Cancer 6(1): 23-28 (1999).

34. Tang et al., J. Cellular Biochem., 50: 350-56 (1992).

What is claimed is:

1. A method for treating excessive osteolysis in a patient, comprising administering to said patient an effective amount of a compound of Formula I: 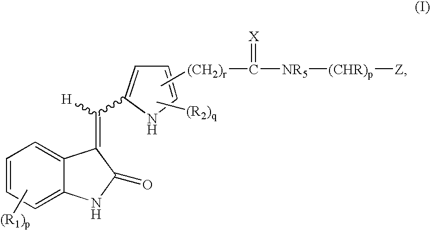
wherein
R is independently H, OH, alkyl, aryl, cycloalkyl, heteroaryl, alkoxy, heterocyclic and amino;
each R₁ is independently selected from the group consisting of alkyl, halo, aryl, alkoxy, haloalkyl, haloalkoxy, cycloalkyl, heteroaryl, heterocyclic, hydroxy, —C(O)—R₈, —NR₉R₁₀, —NR₉C(O)—R₁₂ and —C(O)NR₉R₁₀;
each R₂ is independently selected from the group consisting of alkyl, aryl, heteroaryl, —C(O)—R₈ and SO₂R″, where R″ is alkyl, aryl, heteroaryl, NR₉N₁₀ or alkoxy;
each R₅ is independently selected from the group consisting of hydrogen, alkyl, aryl, haloalkyl, cycloalkyl, heteroaryl, heterocyclic, hydroxy, —C(O)—R₈ and (CHR)ᵣR₁₁;
X is O or S;
p is 0-3;
q is 0-2;
r is 0-3;
R₈ is selected from the group consisting of —OH, alkyl, aryl, heteroaryl, alkoxy, cycloalkyl and heterocyclic;
R₉ and R₁₀ are independently selected from the group consisting of H, alkyl, aryl, aminoalkyl, heteroaryl, cycloalkyl and heterocyclic, or R₉ and R₁₀ together with N may form a ring, where the ring atoms are selected from the group consisting of C, N, O and S;
R₁₁ is selected from the group consisting of —OH, amino, monosubstituted amino, disubstituted amino, alkyl, aryl, heteroaryl, alkoxy, cycloalkyl and heterocyclic;
R₁₂ is selected from the group consisting of alkyl, aryl, heteroaryl, alkoxy, cycloalkyl and heterocyclic;
Z is OH, O-alkyl, or —NR₃R₄, where R₃ and R₄ are independently selected from the group consisting of hydrogen, alkyl, aryl, heteroaryl, cycloalkyl, and heterocyclic, or
R₃ and R₄ may combine with N to form a ring where the ring atoms are selected from the group consisting of CH₂, N, O and S or 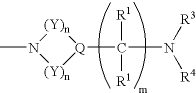
wherein Y is independently CH₂, O, N or S,
Q is C or N;
n is independently 0-4; and
m is 0-3;
or a salt thereof.

2. The method of claim 1, wherein R₁ is halo and p is 1.

3. The method of claim 2, where Z is —NR₃R₄, wherein R₃ and R₄ form a morpholine ring.

4. The method of claim 1, wherein Z is: 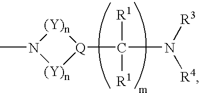
wherein each Y is CH₂, each n is 2, m is 0 and R₃ and R₄ form a morpholine ring.

5. The method of any of claims 1-3, wherein R₂ is methyl and q is 2, wherein the methyls are bonded at the 3 and 5 positions.

6. The method of claim 1, wherein the compound administered is a compound of Formula II:

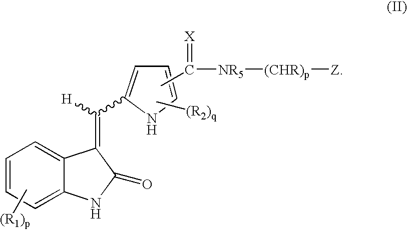

7. The method of claim 6, wherein R₅ is H.

8. The method of claim 6, wherein R₂ is methyl, q is 2, wherein the methyls are bonded at the 3 and 5 positions.

9. The method of claim 6, wherein the patient has cancer that has metastasized to bone.

10. The method of claim 6, wherein the patient has a cancer that secretes M-CSF.

11. The method of claim 6, wherein the patient has osteoporosis.

12. The method of claim 6, wherein the patient is post-menopausal.

13. The method of claim 1, wherein the compound administered is selected from the group consisting of

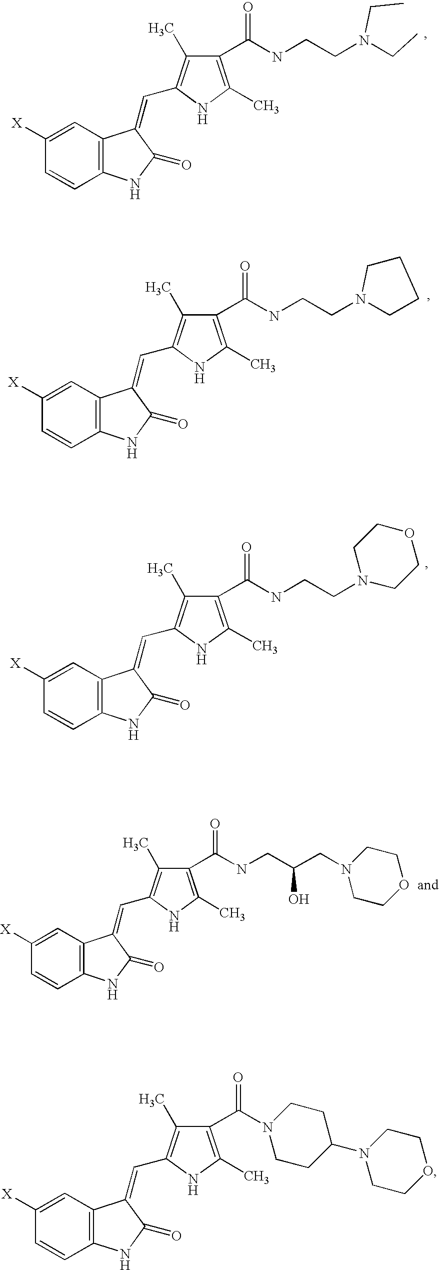

14. The method of claim 1, wherein the compound of formula I is selected from the group consisting of:

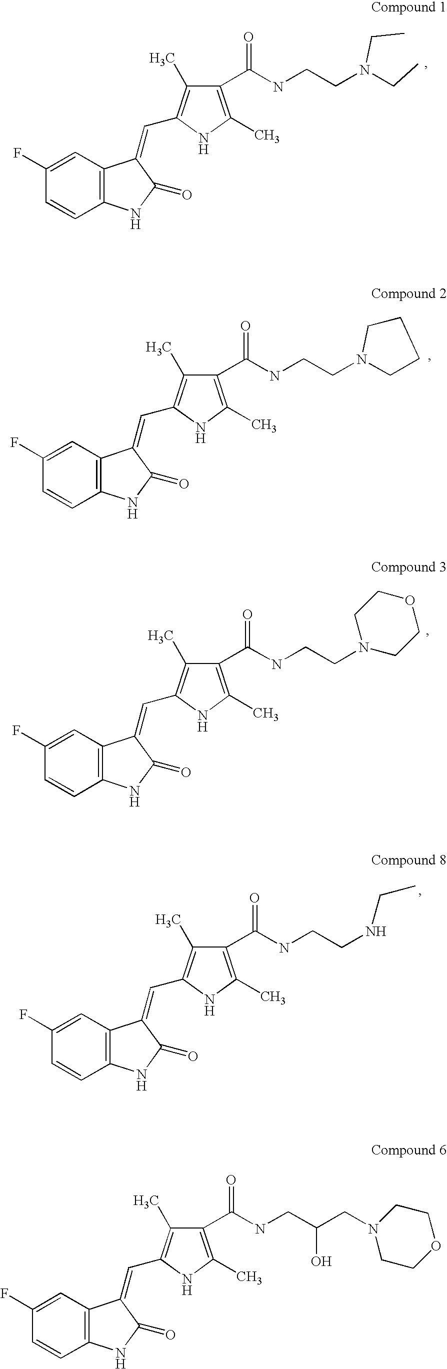

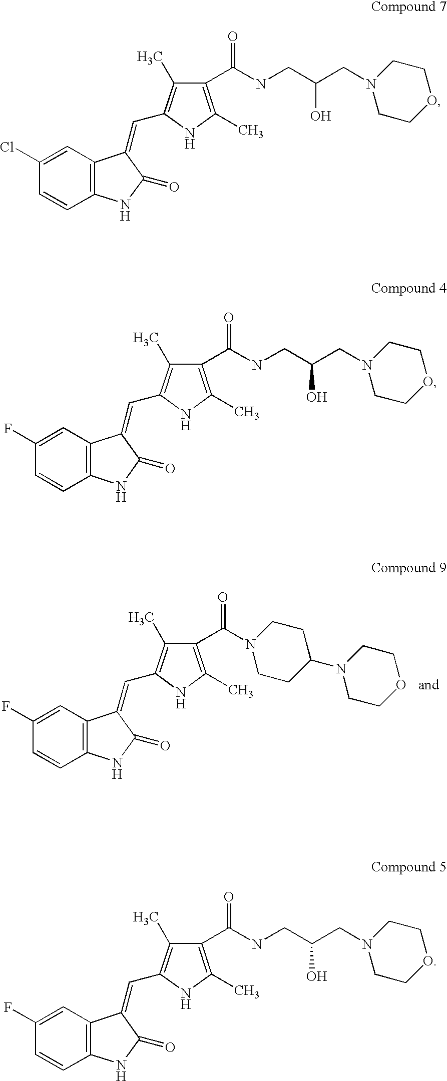

15. A method of inhibiting phosphorylation of CSF 1 R in a patient in need of such inhibition, comprising administering to said patient an inhibitory amount of a compound of Formula I: 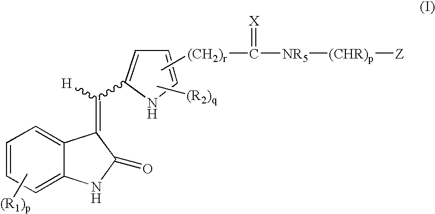
wherein
R is independently H, OH, alkyl, aryl, cycloalkyl, heteroaryl, alkoxy, heterocyclic and amino;
each R₁ is independently selected from the group consisting of alkyl, halo, aryl, alkoxy, haloalkyl, haloalkoxy, cycloalkyl, heteroaryl, heterocyclic, hydroxy, —C(O)—R₈, —NR₉R₁₀, —NR₉C(O)—R₁₂ and —C(O)NR₉R₁₀;
each R₂ is independently selected from the group consisting of alkyl, aryl, heteroaryl, —C(O)—R₈ and SO₂R″, where R″ is alkyl, aryl, heteroaryl, NR₉N₁₀ or alkoxy;
each R₅ is independently selected from the group consisting of hydrogen, alkyl, aryl, haloalkyl, cycloalkyl, heteroaryl, heterocyclic, hydroxy, —C(O)—R₈ and (CHR)ᵣR₁₁;
p is 0-3;
q is 0-2;
r is 0-3;
R₈ is selected from the group consisting of —OH, alkyl, aryl, heteroaryl, alkoxy, cycloalkyl and heterocyclic;
R₉ and R₁₀ are independently selected from the group consisting of H, alkyl, aryl, aminoalkyl, heteroaryl, cycloalkyl and heterocyclic, or R₉ and R₁₀ together with N may form a ring, where the ring atoms are selected from the group consisting of C, N, O and S;
R₁₁ is selected from the group consisting of —OH, amino, monosubstituted amino, disubstituted amino, alkyl, aryl, heteroaryl, alkoxy, cycloalkyl and heterocyclic
R₁₂ is selected from the group consisting of alkyl, aryl, heteroaryl, alkoxy, cycloalkyl and heterocyclic;
Z is OH, O-alkyl, or —NR₃R₄, where R₃ and R₄ are independently selected from the group consisting of hydrogen, alkyl, aryl, heteroaryl, cycloalkyl, and heterocyclic, or R₃ and R₄ may combine with N to form a ring where the ring atoms are selected from the group consisting of CH₂, N, O and S or 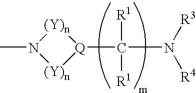
wherein Y is independently CH₂, O, N or S,
Q is C or N
n is independently 0-4; and
m is 0-3.
